(19) United States
(12) Patent Application Publication
(10) Pub. No.: US 2021/0177880 A1
(43) Pub. Date: Jun. 17, 2021

(54) METHODS AND COMPOSITIONS FOR TREATING ACUTE MYELOID LEUKEMIA

(71) Applicants: President and Fellows of Harvard College, Cambridge, MA (US); The General Hospital Corporation, Boston, MA (US)

(72) Inventors: Nick Van Gastel, Cambridge, MA (US); David T. Scadden, Weston, MA (US); Toshihiko Oki, Brookline, MA (US); Amir Schajnovitz, Cambridge, MA (US)

(21) Appl. No.: 16/760,901

(22) PCT Filed: Oct. 31, 2018
(86) PCT No.: PCT/US18/58589

§ 371 Date: Apr. 30, 2020

Related U.S. Application Data
(60) Provisional application No. 62/579,843, filed on Oct. 31, 2017.
(60) Provisional application No. 62/596,749, filed on Dec. 8, 2017.
(60) Provisional application No. 62/639,782, filed on Mar. 7, 2018.
(60) Provisional application No. 62/651,150, filed on Mar. 31, 2018.

(51) Int. Cl. A61K 31/7048

(52) U.S. Cl. CPC A61K 31/7048

(57) ABSTRACT

The disclosure relates to compositions, methods, and kits for treating leukemia, specifically acute myeloid leukemia, in a subject, and for detecting chemoresistant acute myeloid leukemic cells.

### CROSS-REFERENCE TO RELATED APPLICATIONS

This application claims the benefit of U.S. Provisional Application No. 62/579,843, filed on Oct. 31, 2017, U.S. Provisional Application No. 62/596,749, filed on Dec. 8, 2017, U.S. Provisional Application No. 62/639,782, filed on Mar. 7, 2018, and U.S. Provisional Application No. 62/651,150, filed on Mar. 31, 2018. The entire teachings of the above applications are incorporated herein by reference.

### BACKGROUND OF THE INVENTION

Acute myeloid leukemia (AML) is a malignancy of hematopoietic stem and progenitor cells that annually affects 20,000 people and claims 13,000 lives in the US alone (National Comprehensive Cancer Network (NCCN), Clinical Practice Guidelines in Oncology (2016)). In recent decades, great strides have been made in understanding the cytogenetic changes and genetic mutations associated with AML. New therapeutic strategies however have not yet been realized and the survival of AML patients has not improved significantly in decades (Clinical Practice Guidelines in Oncology; Howlader N, et al., SEER Cancer Statistics Review (2016)).

Conventional AML therapy is based on intensive use of cytarabine or other nucleoside analogs in combination with anthracyclines such as daunorubicin or doxorubicin (Clinical Practice Guidelines in Oncology). Although this induction chemotherapy (iCT) regimen will induce complete remission in the majority of patients, relapse rates are very high (De Kouchkovsky I, et al. Blood Cancer J (2016) 6:e441). Relapsed AML is associated with high morbidity and mortality, often leaving only hematopoietic stem cell transplantation as available therapeutic option. Preventing relapse is therefore a key challenge in the treatment of AML.

Despite the clear clinical need to prevent or delay relapse, it remains unclear how certain AML cells manage to survive the extreme stress of chemotherapy. Researchers have tried to understand the origins of relapse by looking for specific mutations that would confer chemoresistance in a subset of cells. However, the mutational landscape of AML is complex (Ding et al. Nature (2012) 481:506-510), and recent efforts in genomic profiling have failed to establish a clear link between genetic lesions and chemotherapy resistance (Magee J A, Br J Haematol (2017) 176:5-6). While mutations may certainly play a role, cells have other systems to protect them from stress, such as shifting metabolic programs (Kultz D Annu Rev Physiol (2005) 67:225-257; Naviaux R K Mitochondrion (2014) 16:7-17). Cancer cells are known to have particular metabolic demands, but the idea that chemoresistance in cancer could be driven by metabolic alterations has thus far received little attention (Zhao Y, et al. Cell Death Dis (2013) 4:e532).

### SUMMARY OF THE INVENTION

A novel treatment strategy for AML is described, in which inhibition of glutamine metabolism in combination with iCT leads to unexpected and synergistic induction of cell death. In addition, expression of several glutamine transporters by AML cells strongly increases after chemotherapy, and may be useful as biomarkers to identify residual, chemoresistant AML cells in the bone marrow.

In certain aspects, the inventions disclosed herein relate to methods of targeting chemoresistant acute myeloid leukemia cells in a subject in need thereof, the method comprising administering to the subject an effective amount of a glutamine metabolism inhibitor and an induction chemotherapy treatment regimen, thereby targeting the chemoresistant acute myeloid leukemia cells in the subject.

In some aspects, the inventions disclosed herein relate to methods of treating acute myeloid leukemia in a subject in need thereof. The method comprises administering to the subject an effective amount of a glutamine metabolism inhibitor and an induction chemotherapy treatment (iCT) regimen, thereby treating acute myeloid leukemia in the subject, wherein the iCT regimen comprises administering cytarabine and doxorubicin to the subject for a period of 3 days, followed by administering cytarabine alone to the subject for a period of 2 days, and wherein the glutamine metabolism inhibitor is administered for a period of at least 5 days beginning the day after completing the iCT regimen.

In still other aspects, the inventions disclosed herein relate to methods of treating acute myeloid leukemia in a subject in need thereof. The method comprises administering to the subject an effective amount of a glutamine metabolism inhibitor and an induction chemotherapy treatment regimen, thereby treating acute myeloid leukemia in the subject, wherein the iCT regimen comprises administering cytarabine and doxorubicin to the subject for a period of 3 days, followed by administering cytarabine alone to the subject for a period of 2 days, and wherein the glutamine metabolism inhibitor is administered for a period of at least 5 days with administration beginning 4 days after starting the iCT regimen.

In some aspects, the inventions disclosed herein related to methods of promoting survival of a subject suffering from acute myeloid leukemia. The method comprises administering to the subject an effective amount of a glutamine metabolism inhibitor and an induction chemotherapy treatment regimen, thereby promoting survival of the subject, wherein the iCT regimen comprises administering cytarabine and doxorubicin to the subject for a period of 3 days, followed by administering cytarabine alone to the subject for a period of 2 days, and wherein the glutamine metabolism inhibitor is administered for a period of at least 5 days beginning the day after completing the iCT regimen.

In still other aspects, the inventions disclosed herein related to methods of promoting survival of a subject suffering from acute myeloid leukemia. The method comprises administering to the subject an effective amount of a glutamine metabolism inhibitor and an induction chemotherapy treatment regimen, thereby promoting survival of the subject, wherein the iCT regimen comprises administering cytarabine and doxorubicin to the subject for a period of 3 days, followed by administering cytarabine alone to the subject for a period of 2 days, and wherein the glutamine metabolism inhibitor is administered for a period of at least 5 days with administration beginning 4 days after starting the iCT regimen.

In some embodiments, the glutamine metabolism inhibitor is administered every other day. In some embodiments the glutamine metabolism inhibitor is administered for a period of at least 10 days.

In some embodiments, the glutamine metabolism inhibitor comprises a small molecule inhibitor. In some embodiments the glutamine metabolism inhibitor comprises a glutaminase inhibitor. In some aspects a glutaminase inhibitor comprises a GSL1 inhibitor and/or a GSL2 inhibitor. In some aspects a glutaminase inhibitor comprises 6-diano-5-oxo-L-norleucine (DON) or an analog thereof. In some embodiments the glutamine metabolism inhibitor comprises a solute carrier family 38 member 1 (SLC38a1) inhibitor. In some embodiments the glutamine metabolism inhibitor comprises a solute carrier family 38 member 2 (SLC38a2) inhibitor. In some embodiments the glutamine metabolism inhibitor comprises glutamate-cysteine ligase (GCL) inhibitor. In some embodiments the glutamine metabolism inhibitor comprises a solute carrier family 7 member 11 (SLC7A11) inhibitor. In some embodiments the glutamine metabolism inhibitor comprises dihydroorotate dehydrogenase (DHODH) inhibitor.

The administration of the glutamine metabolism inhibitor and the induction chemotherapy treatment regimen may result in reduced expression of one or more glutamine transporters (e.g., Slc5a1, Slc38a1, and Slc38a2), as compared to administering only induction chemotherapy, or reduction in the number of cells expressing one or more of said glutamine transporters.

In some embodiments, the subject is suffering from refractory or relapsed acute myeloid leukemia. In certain embodiments, the method further comprises evaluating the subject to determine if the subject has refractory or relapsed acute myeloid leukemia. In some embodiments, the subject is a subject who has relapsed from complete remission of acute myeloid leukemia after induction chemotherapy. In certain embodiments, treating acute myeloid leukemia comprises inducing complete remission of acute myeloid leukemia in the subject. Treating acute myeloid leukemia may comprise inducing complete remission of acute myeloid leukemia in the subject in the absence of a relapse risk due to residual leukemic cells in the subject's bone marrow or peripheral blood.

In certain aspects, the inventions disclosed herein relate to methods of detecting a gene expression signature comprising increased expression levels of one or more glutamine transporters in a subject, comprising obtaining a sample from the subject; and detecting whether the gene signature is present in the sample. The glutamine transporters can be selected from the group consisting of Slc5a1, Slc38a1 and Slc38a2, for example. Presence of increased expression levels of one or more glutamine transporters is indicative of the presence of chemoresistant AML cells.

In certain aspects, the inventions disclosed herein relate to methods of detecting a Slc5a1 gene signature or Slc5a1 gene expression in a subject, comprising obtaining a sample from the subject; and detecting whether the Slc5a1 gene signature or gene expression is present in the sample.

In other aspects, the inventions disclosed herein relate to methods of detecting a Slc38a1 gene signature or SLc38a1 gene expression in a subject, comprising obtaining a sample from the subject; and detecting whether the Slc38a1 gene signature or gene expression is present in the sample.

In still other aspects, the inventions disclosed herein relate to methods of detecting a Slc38a2 gene signature or Slc38a2 gene expression in a subject, comprising obtaining a sample from the subject; and detecting whether the Slc38a2 gene signature or expression is present in the sample.

In certain aspects, the inventions disclosed herein relate to methods of detecting chemoresistant AML cells in a subject, comprising obtaining a sample from the subject and detecting one or more gene signatures in a sample, wherein the one or more gene signatures is selected from the group consisting of Slc5a1, Slc38a1, or Slc38a2, and wherein the presence of the gene signature indicates the presence of chemoresistant AML cells.

In still other aspects, the inventions disclosed herein relate to methods of detecting chemoresistant AML cells in a subject, comprising: (a) obtaining a biological sample from a subject treated with chemotherapy; (b) conducting at least one flow panel assay on the sample to detect the level or activity of one or more gene signatures of Slc5a1, Slc38a1, or Slc38a2; and (c) measuring the level of the one or more gene signatures of Slc5a1, Slc38a1, or Slc38a2.

In certain aspects, the inventions disclosed herein relate to methods of targeting chemoresistant acute myeloid leukemia cells in a subject, comprising administering to the subject an effective amount of a glutamine metabolism inhibitor and an induction chemotherapy treatment regimen, thereby targeting the chemoresistant acute myeloid leukemia cells in the subject.

The methods of treatment and methods of detection of chemoresistant AML cells described herein can be used in concert (sequentially, including in repetitive sequence such as treat, detect, treat again, detect again), or each of the methods can be used separately and in combination with other methods known in the art.

In certain aspects, the inventions disclosed herein relate to pharmaceutical compositions comprising an effective amount of a glutamine metabolism inhibitor, an effective amount of at least one chemotherapeutic agent to which a subject having acute myeloid leukemia may be or may become resistant or refractory, and a pharmaceutically acceptable carrier, diluent, or excipient.

In some embodiments, the at least one chemotherapeutic agent is one to which acute myeloid leukemic cells in a patient are or become resistant. In one embodiment the at least one chemotherapeutic agent comprises an antimetabolite agent (e.g., cytarabine). In some embodiments, the at least one chemotherapeutic agent comprises an anthracycline agent (e.g., doxorubicin). In certain embodiments, the at least one chemotherapeutic agent comprises an antimetabolite agent and anthracycline agent (e.g., cytarabine and doxorubicin).

In some embodiments, the glutamine metabolism inhibitor comprises 6-diano-5-oxo-L-norleucine (DON) or an analog thereof.

In certain aspects, the inventions disclosed herein relate to kits comprising a glutamine metabolism inhibitor, at least one chemotherapeutic agent, and instructions for administering the glutamine metabolism inhibitor and the at least one chemotherapeutic agent to a subject suffering from acute myeloid leukemia.

In some embodiments, the instructions further comprise directions for administering the at least one chemotherapeutic agent as part of an induction chemotherapy treatment regimen for the subject. In certain embodiments, the instructions further comprise directions for administering the glutamine metabolism inhibitor, and the at least one therapeutic agent to induce complete remission of acute myeloid leukemia in the subject (e.g., without risk of relapse by completely eradicating leukemic cells in the subject).

In some embodiments, the at least one chemotherapeutic agent comprises an antimetabolite agent (e.g., cytarabine). In some embodiments, the at least one chemotherapeutic agent comprises an anthracycline agent (e.g., doxorubicin). In certain embodiments, the at least one chemotherapeutic agent comprises an antimetabolite agent and an anthracycline agent (e.g., cytarabine and doxorubicin).

In some embodiments, the glutamine metabolism inhibitor comprises a small molecule inhibitor (e.g., 6-diano-5-oxo-L-norleucine (DON) or analogs thereof).

The above discussed and many other features and attendant advantages of the present invention will become better understood by reference to the following detailed description of the invention when taken in conjunction with the accompanying examples.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 demonstrates untargeted metabolomics analysis of AML cells. Heatmap-based visualization (logarithmic scale) of the 35 most significantly different metabolites between cells isolated from AML-bearing mice with vehicle treatment (n=4), at 3 days after iCT (iCT; n=10) or at 10 days after iCT (relapse; n=3). Unsupervised clustering was performed on normalized data using the Ward clustering algorithm. Metabolites involved in glutamine metabolism are indicated in orange.

FIG. 2 demonstrates untargeted metabolomics analysis of AML cells: validation. Heatmap-based visualization (logarithmic scale) of the 20 most significantly different metabolites between cells isolated from AML-bearing mice with vehicle treatment (n=5), at 3 days after iCT (iCT; n=6) or at 10 days after iCT (relapse; n=5). Unsupervised clustering was performed on normalized data using the Ward clustering algorithm. Metabolites involved in glutamine metabolism are indicated in orange.

FIG. 3 demonstrates levels of individual metabolites in AML cells after vehicle or iCT treatment. Mass spectrometry based quantification of metabolites involved in glutamine metabolism in AML cells isolated from the bone marrow of mice treated with vehicle or iCT (3 days after last dose).

FIGS. 4A-4E demonstrate overexpression of glutamine transporters in chemoresistant AML cells. Chemoresistant AML cells have increased levels of glutamine transporters but unchanged levels of glutamine metabolism enzymes. FIGS. 4A-4B show relative gene expression levels of enzymes involved in glutamine metabolism (FIG. 4A) and glutamine transporters (FIG. 4B) obtained through RNA sequencing of AML cells from vehicle- or iCT-treated mice. FIG. 4C shows SLC38A1 protein levels in AML cells (GFP+) and normal hematopoietic cells (GFP−) from vehicle- or iCT-treated mice. FIG. 4D provides a schematic overview of glutamine metabolism enzymes and glutamine transporters. FIG. 4E shows AML patient survival datasets demonstrating that patients who had leukemia with high expression levels of SLC38A1 had a lower probability of survival.

FIGS. 5A-5E demonstrate inhibition of glutamine metabolism increases response to chemotherapy in AML. FIG. 5A shows viability of human AML cells (THP1 cell line) after 72 h of in vitro treatment with different concentrations of iCT and DON. Arrows show the range where synergism occurs. FIG. 5B shows survival of mice treated with iCT (cytarabine 100 mg/kg once daily for 5 days+doxorubicin 3 mg/kg once daily for first 3 days), DON (0.3 mg/kg once daily for 5 days), or the combination. FIG. 5C provides the chemical structure of glutamine and 6-diazo-5-oxo-L-norleucine (DON). FIGS. 5D-5E show survival of AML-bearing mice treated with iCT (days 7-11) with or without short term 6-diazo-5-oxo-L-norleucine (DON; 0.3 mg/kg), showing the treatment schedule (FIG. 5D) and Kaplan-Meier survival curves (FIG. 5E). iCT+DON: both given at same time; iCT+DON⁺³: DON started 3 days after first dose of iCT. FIGS. 5F-5G show survival of AML-bearing mice treated with iCT (days 7-11) with or without continuous DON (9.3 mg/kg), showing the treatment schedule (FIG. 5F) and Kaplan-Meier survival curves (FIG. 5G). iCT+DON^{post}: DON started after last dose of iCT.

FIGS. 6A-6B demonstrate the identification of the moment of maximal response to chemotherapy in a mouse model of aggressive AML. The moment of maximal response to chemotherapy occurs 3-4 days after the last dose of chemotherapy. FIG. 6A provides a schematic of the protocol for infecting and treating mice whose cells express MLL-AF9, luciferase, and GFP. FIG. 6B shows disease progression visualized by bioluminescence imaging of the experimental mice injected with 1×10⁶ AML cells at day 0, treated with a chemotherapy regimen that closely mimics the one used in patients (cytarabine for 5 days+doxorubicin for the first 3 days) (right panel) or vehicle (left panel). Arrow indicates the moment of maximal response.

FIGS. 7A-7D demonstrate metabolic profiling of AML cells after chemotherapy. The metabolic profile of AML cells freshly isolated from the bone marrow of mice treated with vehicle (vehicle group) or treated with iCT, both at 3 days after the last dose (iCT group) or at 10 days after the last dose (relapse group) was identified. FIG. 7A shows two independent experiments were performed, and provides the overlap between both experiments and the number of metabolites that could be putatively identified. Metabolites detected in both experiments were used for subsequence analysis, with 61 metabolites being annotated and 39 metabolites unknown. FIGS. 7B-7C show principal component analysis (FIG. 7B) and heatmap-based visualization (FIG. 7C) of the metabolic profile of AML cells obtained from mice with vehicle treatment (vehicle group), at 3 days after iCT (iCT group) or at 10 days after iCT (relapse group). FIG. 7B shows separation of the vehicle group from the iCT and relapse groups. FIG. 7C provides sets of metabolites exhibiting different patterns between the three groups. Untargeted metabolomics analysis of chemoresistant AML cells reveals changes in glutamine metabolism. FIG. 7D shows 360 metabolites were detected after analysis of 25,000 cells, of which 216 (60%) could be putatively identified. One pathway stood out: glutamine metabolism, with 4 metabolites (glutamine, glutamate, pyroglutamate, and aspartate) being significantly increased in the chemoresistant cells.

FIGS. 8A-8C demonstrate optimization of untargeted metabolomics analysis of freshly isolated AML cells. Untargeted metabolomics analysis of chemoresistant AML cells reveals changes in glutamine metabolism. FIG. 8A provides a schematic overview of cell isolation and sample processing for untargeted metabolomics of freshly isolated AML cells, including dissecting and crushing long bones to isolate GFP⁺ AML cells using FACS. Cells were then lysed and polar metabolites were obtained after methanol:chloroform extraction. FIG. 8B shows analysis of the effect of FACS sorting on the cellular metabolome profile of AML cells, showing peak area distribution (measure for total metabolite levels; left panel) and correlation of individual metabolite levels (right panel) between unsorted and FACS sorted cells, obtained from in vitro culture. AML cells were either used for immediate metabolite extraction (unsorted), or subjected to incubation at 4° C. and FACS sorting prior to metabolite extraction (FACS sorted). FIG. 8C shows the levels of different metabolites that can be detected when using increasing amounts of cells as starting material for mass spectrometry analysis. The results are of a dose-response experiment, which showed that while some metabolites were detectable at lower cell numbers, 500,000 cells are needed for the detection of several metabolites involved in central carbon metabolism.

FIGS. 9A-9C demonstrate pathway enrichment analysis reveals differences in glutamine metabolism. FIG. 9A shows Metabolite Sets Enrichment Analysis of the 61 annotated meatbolites of the vehicle and iCT groups (FIG. 7A) showing metabolic pathways enriched in AML cells after iCT treatment. FIG. 9B shows levels of individual metabolites related to glutamine metabolism. *p<0.05, **p<0.01, ***p<0.001, ****p<0.0001. The top four pathways all contained the same 3 metabolites that drove the enrichment and statistical significance: glutamine, glutamate and aspartate. The levels of these metabolites in the three groups exhibited a similar pattern: low in the vehicle group, high in the iCT group, and low again in the relapse group. TCA cycle metabolites showed overall less differences between the groups, although succinate levels were increased while citrate/isocitrate levels were decreased in chemoresistant AML cells. FIG. 9C provides a schematic representation of glutamine metabolism and the tricarboxylic acid (TCA) cycle.

### DETAILED DESCRIPTION OF THE INVENTION

The disclosure relates to the discovery of a novel treatment strategy for leukemia (e.g., acute myeloid leukemia (AML)), in which inhibition of glutamine metabolism in combination with chemotherapy (e.g., standard induction chemotherapy (cytarabine+doxorubicin)) leads to unexpected and synergistic induction of leukemia cell death. It was found that glutamine metabolism inhibitors can overcome resistance to standard chemotherapy in AML. In addition, expression of several glutamine transporters by AML, cells strongly increases after chemotherapy, and may be useful as biomarkers to identify residual, chemoresistant AML cells in the bone marrow. Accordingly, the disclosure contemplates the use of one or more agents (e.g., glutamine metabolism inhibitors) in methods, compositions, and kits for treating AML.

Targeting Chemoresistant Cells

In some aspects, disclosed herein are methods for targeting chemoresistant leukemic cells in a population of cells. Such methods are useful for, amongst other things, treating leukemia (e.g., acute myeloid leukemia). In one embodiment, a method of targeting chemoresistant leukemic cells in a population of cells comprises contacting the population of cells with an effective amount of a glutamine metabolism inhibitor in combination with an induction chemotherapy treatment regimen, thereby targeting chemoresistant leukemic cells in the cell population.

It should be appreciated by those skilled in the art that the compositions and methods described herein decrease the amount or activity of leukemic cells in a population of cells. In some embodiments, the compositions and methods described herein preferably decrease the number, activity, and/or proliferation of chemoresistant leukemic cells in a population of cells. The amount or number of leukemic cells eradicated, reduced, or inhibited in any particular population of cells can be proportional to the concentration of glutamine metabolism inhibitor to which the population of cells has been exposed. In some instances, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100% of the leukemic cells in the population of cells are eradicated, reduced, or inhibited by exposure to or contact with a glutamine metabolism inhibitor in combination with an induction chemotherapy regimen. In some embodiments, at least 20% of the leukemic cells in the population of cells are eradicated, reduced, or inhibited. In some embodiments, at least 50% of the leukemic cells in the population of cells are eradicated, reduced, or inhibited. In some embodiments, at least 70% of the leukemic cells in the population of cells are eradicated, reduced, or inhibited. In some embodiments, all of the leukemic cells in the population of cells are eradicated, reduced, or inhibited. In certain embodiments, the leukemic cells are chemoresistant leukemic cells.

In some embodiments, the targeting of chemoresistant cells results in reduced mRNA expression of one or more glutamine transporters. In some aspects, the one or more glutamine transporters are selected from the group consisting of Slc5a1, Slc38a1, and Slc38a2. Expression levels of the one or more glutamine transporters can be compared between cells (e.g., AML cells) treated with a glutamine metabolism inhibitor in combination with induction chemotherapy and cells treated with only induction chemotherapy.

The present invention contemplates eradicating leukemic cells by contacting a population of cells with, or exposing the population of cells to, a glutamine metabolism inhibitor in combination with an induction chemotherapy regimen. In some embodiments, the leukemia cells comprise leukemia cells from an acute myeloid leukemia cell line. Exemplary acute myeloid leukemia cell lines include, but are not limited to, MLL-AF9 cells, MLL-ENL cells, Nup98-HoxA9 cells, AML1-ETO9A cells, KG-1 cells, KG-1a cells, U937 cells, THP1 cells, HL60 cells, HoxA9/Meis1 cells, and NB-4 cells. In some embodiments, the population of cells comprises primary leukocytes, such as bone marrow leukocytes and peripheral blood leukocytes. Examples of such primary leukocytes include, without limitation, stem and progenitors, mononuclear cells, myeloblasts, neutrophils, NK cells, macrophages, granulocytes, monocytes, and lineage-/cKit+/Sca1+ (LKS) cells.

In some aspects a glutamine metabolism inhibitor comprises a small molecule, nucleic acid, polypeptide, peptide, drug, ion, etc. An “inhibitor” can be any chemical, entity or moiety, including without limitation synthetic and naturally-occurring proteinaceous and non-proteinaceous entities. In some embodiments, an inhibitor is nucleic acids, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, inhibitors are a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

In some aspects a glutamine metabolism inhibitor is a glutaminase inhibitor. Glutaminase comprises a “kidney-type” (GLS1) and a “liver-type” (GLS2). In some aspects a glutaminase inhibitor inhibits, partially or completely, the conversion of glutamine into glutamate. In certain aspects a glutaminase inhibitor is a GLS1 inhibitor and/or a GLS2 inhibitor. In some aspects the glutaminase inhibitor is a siRNA (e.g., a GLS2 or GLS1 siRNA). In some embodiments the glutaminase inhibitor is selected from the group consisting of 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (also known as CB-839), 5-(3-Bromo-4-(dimethylamino)phenyl)-2,2-dimethyl-2,3,5,6-tetrahydrobenzo[a]phenanthridin-4(1H)-one (also known as 968), and N,N′-[Thiobis(2,1-ethanediyl-1,3,4-thiadiazole-5,2-diyl)]bisbenzeneacetamide (also known as BPTES). In some embodiments a glutaminase inhibitor is an alkyl benzoquinone, such as those described by Lee et al., Discovery of selective inhibitors of Glutaminase-2, which inhibit mTORC1, activate autophagy and inhibit proliferation in cancer cells. Oncotarget 5(15): 6087-6101 (2014), incorporated herein by reference. In some embodiments a glutaminase inhibitor is one such as those described by McDermott et al., Design and Evaluation of Novel Glutaminase Inhibitors, Bioorg. Med. Chem. 24:1819-1839 (2016) (e.g., compound 7c (UPGL00004)), incorporated herein by reference. In some embodiments a glutaminase inhibitor is 6-diazo-5-oxo-L-norleucine (also known as DON).

In some aspects a glutamine metabolism inhibitor is a solute carrier family 38 member 1 (SLC38a1) and/or solute carrier family 38 member 2 (SLC38a2) inhibitor. In some embodiments a SLC38a1 and/or SLC38a2 inhibitor is selected from the group consisting of lithium, potassium choline, N-methyl-D-glucamine, and 2-methylamino-isobutyric acid (MeAIB).

In some aspects a glutamine metabolism inhibitor is a glutamate-cysteine ligase (GCL) inhibitor. In some embodiments a GCL inhibitor is 1-buthionine-[S,R]-sulfoximine (BSO). In some embodiments a GCL inhibitor is glutathione.

In some aspects a glutamine metabolism inhibitor is a solute carrier family 7 member 11 (SLC7A11) inhibitor. In some embodiments a SLC7A11 inhibitor is glutamate. In some embodiments a SLC7A11 inhibitor is selected from the group consisting of erastin, sulfasalazine, and sorafenib.

In some aspects a glutamine metabolism inhibitor is a dihydroorotate dehydrogenase (DHODH) inhibitor. In some embodiments a DHODH inhibitor is an selected from the group consisting of teriflunomide, leflunomide, and brequinar sodium (BRQ). In some embodiments a DHODH inhibitor is one such as those described by Sykes et al., Inhibition of Dihydroorotate Dehydrogenase Overcomes Differentiation Blockade in Acute Myeloid Leukemia, Cell, 167:171-186 (2016), incorporated herein by reference. In some embodiments a DHODH inhibitor is one such as those described by Lolli et al., Use of human Dihydroorotate Dehydrogenase (hDODH) Inhibitors in Autoimmune Diseases and New Perspectives in Cancer Therapy, Recent patents on Anti-Cancer Drug Discovery, 13(1):86-105 (2018), incorporated herein by reference.

It should be appreciated that the effective amount of the agents for use in accordance with the present inventions (e.g., a glutamine metabolism inhibitor) may vary, for example, depending on the glutamine metabolism inhibitor being used and its location of use. In some embodiments, the effective amount of the glutamine metabolism inhibitor for in vitro use comprises a concentration in the range of 0.01 μM to 500 μM, or alternatively within the range of 0.5 μM to 1.0 μM. In some embodiments, the effective amount of the glutamine metabolism inhibitor for in vivo use comprises a concentration in the range of 0.10 mg/kg to 5.0 mg/kg, or within the range of 0.25 mg/kg to 1.0 mg/kg. In some embodiments, the effective amount comprises a concentration of 0.25 mg/kg. In some embodiments, the effective amount comprises a concentration of 0.30 mg/kg. In some embodiments, the effective amount comprises a concentration of 0.35 mg/kg.

It is generally understood that synergism may occur between a glutamine metabolism inhibitor and the induction chemotherapy treatment for effectively targeting and treating chemoresistant AML cells. In some embodiments, the point of synergism between a glutamine metabolism inhibitor (e.g., DON) and induction chemotherapy (e.g., cytarabine and/or doxorubicin) may vary depending on the type of glutamine metabolism inhibitor used and the specific induction chemotherapy treatment. In some embodiments, synergism occurs for an in vitro treatment when the induction chemotherapy treatment comprises cytarabine in an amount between 10⁻³ and 10⁻² μg/ml and the glutamine metabolism inhibitor comprises DON in an amount of 0.8 μM.

In some embodiments, the contacting occurs in vitro or ex vivo. In other embodiments, the contacting occurs in vivo. In some embodiments, the in vivo contact is in a subject as described herein.

Methods of Treatment

The disclosure contemplates various methods of treatment utilizing the compositions and kits comprising the glutamine metabolism inhibitors and induction chemotherapy treatments described herein. The disclosure contemplates the treatment of any disease in which cells are chemoresistant. The glutamine metabolism inhibitors described herein can be used to treat and/or prevent such diseases.

In some aspects, the disclosure provides a method of treating acute myeloid leukemia in a subject in need thereof, the method comprising administering to the subject an effective amount of a glutamine metabolism inhibitor described herein, thereby treating acute myeloid leukemia in the subject. In some embodiments, the method further comprises administering an induction chemotherapy treatment regimen to the subject. The disclosure contemplates administering any induction chemotherapy treatment regimen that is useful for inducing complete remission of acute myeloid leukemia in a subject. In some embodiments, the induction chemotherapy comprises administering an antimetabolite agent (e.g., cytarabine) and an anthracycline agent (e.g., doxorubicin) to the subject. In some embodiments, the antimetabolite agent comprises cytarabine. The induction chemotherapy treatment regimen can be administered to the subject over a period of hours, days, or months. The chemotherapeutic agents used in the induction chemotherapy treatment regimen can be administered at the same time throughout the period, or administered at different intervals within the period. In some embodiments, the induction chemotherapy comprises administering cytarabine and doxorubicin to the subject for a period of 5 days. In some embodiments, the induction chemotherapy comprises administering cytarabine and doxorubicin to the subject for a period of 3 days, followed by administering cytarabine alone to the subject for a period of 2 days.

The glutamine metabolism inhibitor can be administered to the subject before the induction chemotherapy treatment regimen is administered to the subject, at the same time the induction chemotherapy treatment regimen is administered to the subject, after the induction chemotherapy treatment regimen is administered to the subject, or any combination of the above. In some embodiments, the glutamine metabolism inhibitor is administered to the subject for at least a day before administering the induction chemotherapy treatment regimen to the subject. In some embodiments, the glutamine metabolism inhibitor is administered to the subject for at least a day before administering the induction chemotherapy treatment regimen to the subject concomitantly with the glutamine metabolism inhibitor. In some embodiments, the glutamine metabolism inhibitor is administered to the subject at least 2 days, at least 3 days, at least 4 days, at least 5 days, at least 6 days, or up to at least a week before administering the induction chemotherapy treatment regimen to the subject. In some embodiments, the glutamine metabolism inhibitor is administered to the subject at least 8 days, at least 9 days, at least 10 days, at least 11 days, at least 12 days, at least 13 days, at least 2 weeks, at least 3 weeks, or at least a month before the induction chemotherapy treatment regimen is administered to the subject. In some embodiments, the glutamine metabolism inhibitor is administered to the subject for at least 1 day, at least 2 days, at least 3 days, at least 4 days, at least 5 days, at least 6 days, or up to at least a week before administering the induction chemotherapy treatment regimen to the subject, and then the induction chemotherapy regimen is administered to the subject concomitantly with the glutamine metabolism inhibitor for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, or at least a month. In some embodiments, the glutamine metabolism inhibitor is administered to the subject for at least 1 day, at least 2 days, at least 3 days, at least 4 days, at least 5 days, at least 6 days, or up to at least a week before administering the induction chemotherapy treatment regimen to the subject, and then the induction chemotherapy regimen is administered to the subject concomitantly with the glutamine metabolism inhibitor for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, or at least a month, before ceasing administration of the induction chemotherapy regimen while continuing administration of the glutamine metabolism inhibitor to the subject for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, or at least a month. In some embodiments, the glutamine metabolism inhibitor blocker is administered to the subject for at least 2 days before administering an induction chemotherapy treatment regimen comprising 100 mg/kg cytarabine+3 mg/kg doxorubicin to the subject concomitantly with or without administering the glutamine metabolism inhibitor for 3 days, followed by chemotherapy with 100 mg/kg cytarabine in the absence of doxorubicin concomitantly with or without the glutamine metabolism inhibitor for 2 days, followed by 2 weeks (14 days) of administration of the glutamine metabolism inhibitor to the subject. In some embodiments, DON is administered to the subject for at least 2 days before administering an induction chemotherapy treatment regimen comprising 100 mg/kg cytarabine+3 mg/kg doxorubicin to the subject concomitantly with or without administering DON for 3 days, followed by chemotherapy with 100 mg/kg cytarabine in the absence of doxorubicin concomitantly with or without the DON for 2 days, followed by 2 weeks (14 days) of administration of DON to the subject. In some embodiments, administration of the glutamine metabolism inhibitor described herein comprises administering ascending and intermittent concentrations or doses of glutamine metabolism inhibitor described herein over a period of time to the subject. For example, glutamine metabolism inhibitor can be administered at 0.30 mg/kg for at least 1 day, at least 2 days, at least 3 days, at least 4 days, at least 5 days, at least 6 days, or at least a week, followed by at least 1 day, at least 2 days, at least 3 days, at least 4 days, at least 5 days, or at least 1 week in the absence of administering glutamine metabolism inhibitor. It should be appreciated that the concentration or dosage of the glutamine metabolism inhibitor administered initially and, if applicable, at successive intervals after intermission of treatment can vary, as well as the escalation of the concentration or dose between treatment intervals. For example, the initial dose or concentration of the glutamine metabolism inhibitor can be 0.10 mg/kg, 0.15 mg/kg, 0.20 mg/kg, 0.25 mg/kg, 0.30 mg/kg, 0.35 mg/kg, 0.40 mg/kg, 0.45 mg/kg, or 0.50 mg/kg or more, and the escalation of the concentration or dose between intervals can be 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, or 0.10 mg/kg. In addition, ascending and intermittent concentrations of doses of the glutamine metabolism inhibitor can be administered over a variety of treatment intervals, e.g., 2, 3, 4, 5, 6, 7, 8, 9, 10, or as many as desired until the subject enters remission, to keep the subject in remission, or to further prolong survival of the patient, for example, by inducing the patient into remission or preventing the patient from relapsing from remission. In some embodiments, the treatment and intermission from treatment intervals can be more than a week, e.g., 2 weeks, 3 weeks, 4 weeks, 1 month, 2 months, 3 months, 6 months, or a year depending on the course of the disease in the subject. The aforementioned ascending and intermittent concentration or dosing schedules can be used when a subject is at a terminal state of the disease, for example, when leukemic cells are spread all over the subject's body, to prolong survival time of the subject.

As used herein, “treat,” “treatment,” “treating,” or “amelioration” when used in reference to a disease, disorder or medical condition, refers to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of, for example, acute myeloid leukemia, delay or slowing progression of acute myeloid leukemia, and an increased lifespan as compared to that expected in the absence of treatment.

In some embodiments, treating acute myeloid leukemia comprises inducing complete remission of acute myeloid leukemia in the subject. In some embodiments, treating acute myeloid leukemia comprises inducing complete remission of acute myeloid leukemia in the subject in the absence of a relapse risk due to residual leukemic cells in the subject's bone marrow or peripheral blood.

In some embodiments, the method further comprises evaluating the subject to determine if the subject has refractory or relapsed acute myeloid leukemia.

In some embodiments, the administration of the glutamine metabolism inhibitor and the induction chemotherapy treatment regimen results in reduced mRNA levels of one or more glutamine transporters, as compared to administering only induction chemotherapy. In some aspects, the one or more glutamine transporters are selected from the group consisting of Slc5a1, Slc38a1, and Slc38a2.

In some aspects, the disclosure provides a method of promoting survival of a subject suffering from acute myeloid leukemia, the method comprising administering to the subject an effective amount of a glutamine metabolism inhibitor, thereby promoting survival of the subject. The method contemplates any glutamine metabolism inhibitor described herein. In some embodiments, the glutamine metabolism inhibitor comprises 6-diazo-5-oxo-L-norleucine (DON) or an analog thereof.

In some embodiments, the method further comprises administering an induction chemotherapy treatment regimen to the subject. In some embodiments, the induction chemotherapy comprises administering an antimetabolite agent and an anthracycline agent to the subject. In some embodiments, the antimetabolite agent comprises cytarabine. In some embodiments, the anthracycline agent comprises doxorubicin. In some embodiments, the induction chemotherapy comprises administering cytarabine and doxorubicin to the patient for a period of 5 days. In some embodiments, the induction chemotherapy comprises administering cytarabine and doxorubicin to the patient for a period of 3 days, followed by administering cytarabine alone to the patient for a period of 2 days. It should be appreciated that any of the administration or dosing schedules and/or treatment regiments described herein can be used with the method.

In some embodiments, the glutamine metabolism inhibitor is administered to the subject for at least a day before administering the induction chemotherapy treatment regimen to the subject. In some embodiments, the glutamine metabolism inhibitor is administered to the subject for at least a day before administering the induction chemotherapy treatment regimen to the subject concomitantly with the glutamine metabolism inhibitor.

In some embodiments, the method further comprises selecting a subject suffering from or exhibiting a terminal state of acute myeloid leukemia. In some embodiments, the subject has advanced tumor metastasis. In some embodiments, the subject has a high tumor burden.

In some embodiments, the method further comprises selecting a subject suffering from or exhibiting chemoresistant acute myeloid leukemia.

“Survival” refers to the subject remaining alive, and includes overall survival as well as progression free survival. “Overall survival” refers to the subject remaining alive for a defined period of time, such as 1 year, 2 years, 3 years, 4 years, 5 years, etc. from the time of diagnosis or treatment.

“Progression free survival” refers to the subject remaining alive, without the acute myeloid leukemia progressing or getting worse.

“Promoting survival” refers to enhancing one or more aspects of survival in a treated subject relative to an untreated subject (i.e., a subject not treated with a glutamine metabolism inhibitor, such as DON), or relative to a subject treated with an approved chemotherapeutic agent alone in the absence of administration of a glutamine metabolism inhibitor. In some embodiments, the glutamine metabolism inhibitor increases the subject's length of survival compared to the subject's length of survival in the absence of receiving the glutamine metabolism inhibitor. In some embodiments, the glutamine metabolism inhibitor increases the subject's likelihood of survival compared to the subject's likelihood of survival in the absence of receiving the glutamine metabolism inhibitor. In some embodiments, administration of the glutamine metabolism inhibitor (e.g., DON) to the subject increases the subject's overall survival time by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, or more relative to subject's overall survival time in the absence of administration of the glutamine metabolism inhibitor and/or compared to chemotherapy treatment alone. In some embodiments, administration of the glutamine metabolism inhibitor (e.g., DON) to the subject increases the subject's overall survival time by at least 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, or, at least 5 fold or more relative to subject's overall survival time in the absence of administration of the glutamine metabolism inhibitor and/or compared to chemotherapy treatment alone. In some embodiments, administration of the glutamine metabolism inhibitor (e.g., DON) to the subject increases the subject's survival time by 1 day, 5 days, 10 days, 30 days, 2 months, 3 months, 4 months, 5 months, 6 months, 7 months, 8 months, 9 months, 10 months, 11 months, 1 year, 18 months, 2 years, 30 months, 3 years, 40 months, 4 years, 5 years, 6 years, 7 years, 8 years, 9 years, 10 years, 15 years, 20 years, 25 years, 30 years, 35 years, 40 years, 50 years, 55 years, 60 years, 65 years, 70 years, or 75 years or more relative to subject's overall survival time in the absence of administration of the glutamine metabolism inhibitor and/or compared to chemotherapy treatment alone.

In one aspect, the disclosure provides a method of inducing complete remission in a subject having relapsed or refractory acute myeloid leukemia by eradicating chemoresistant leukemic cells in the subject, the method comprising: (a) evaluating the subject to determine if the subject has relapsed or refractory acute myeloid leukemia; (b) administering to the subject a glutamine metabolism inhibitor; and (c) administering to the subject an induction chemotherapy treatment regimen comprising an antimetabolite agent and an anthracycline agent for proscribed periods of time, thereby inducing complete remission in the subject by eradicating chemoresistant leukemic cells in the subject.

Subjects

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. In some embodiments, the subject suffers from acute myeloid leukemia.

In some embodiments, the subject is a patient presenting with acute myeloid leukemia. As used herein, “acute myeloid leukemia” encompasses all forms of acute myeloid leukemia and related neoplasms according to the World Health Organization (WHO) classification of myeloid neoplasms and acute leukemia, including all of the following subgroups in their relapsed or refractory state: Acute myeloid leukemia with recurrent genetic abnormalities, such as AML with t(8;21)(q22;q22); RUNX1-RUNX1T1, AML with inv(16)(p13.1q22) or t(16;16)(p13.1;q22); CBFB-MYH11, AML with t(9;11)(p22;q23); MLLT3-MLL, AML with t(6;9)(p23;q34); DEK-NUP214, AML with inv(3)(q21 q26.2) or t(3;3)(q21;q26.2); RPN1-EVI1, AML (megakaryoblastic) with t(1;22)(p13;q13); RBM15-MKL1, AML with mutated NPM1, AML with mutated CEBPA; AML with myelodysplasia-related changes; therapy-related myeloid neoplasms; AML, not otherwise specified, such as AML with minimal differentiation, AML without maturation, AML with maturation, acute myelomonocytic leukemia, acute monoblastic/monocytic leukemia, acute erythroid leukemia (e.g., pure erythroid leukemia, erythroleukemia, erythroid/myeloid), acute megakaryoblastic leukemia, acute basophilic leukemia, acute panmyelosis with myelofibrosis; myeloid sarcoma; myeloid proliferations related to Down syndrome, such as transient abnormal myelopoiesis or myeloid leukemia associated with Down syndrome; and blastic plasmacytoid dendritic cell neoplasm.

In some embodiments, the methods described herein further comprise selecting a subject diagnosed with acute myeloid leukemia, for example, based on the symptoms presented. Symptoms associated with acute myeloid leukemia are known to the skilled practitioner. For example, a patient can be diagnosed with acute myeloid leukemia if the subject presents with a myeloid neoplasm with 20% or more blasts in the peripheral blood or bone marrow.

In some embodiments, the methods described herein further comprise selecting a subject at risk of developing acute myeloid leukemia. For example, a subject can be selected as at risk of developing leukemia based on a family history of leukemias.

In some embodiments, a subject is selected as diagnosed with acute myeloid leukemia or at risk of developing acute myeloid leukemia based on a genetic mutation useful as a diagnostic or prognostic marker of myeloid neoplasms. Exemplary such markers include mutations of: JAK2, MPL, and KIT in MPN; NRAS, KRAS, NF1, and PTPN11 in MDS/MPN; NPM1, CEBPA, FLT3, RUNX1, KIT, WT1, and MLL in AML; and GATA1 in myeloid proliferations associated with Down syndrome (see Vardiman, et al., “The 2008 revision of the World Health Organization (WHO) classification of myeloid neoplasms and acute leukemia: rationale and important changes,” Blood 114(5), 937-951 (2009), incorporated herein by reference in its entirety).

In some embodiments, the methods described herein further comprise selecting a subject suspected of having acute myeloid leukemia. A subject suspected of having acute myeloid leukemia, for example, can be selected based on family history, diagnostic testing or based on the symptoms presented or a combination thereof.

In some embodiments, the methods described herein further comprise selecting a subject suffering from refractory or relapsed acute myeloid leukemia. As used herein, “relapsed acute myeloid leukemia” is defined as reappearance of leukemic blasts in the blood or greater than 5% blasts in the bone marrow after complete remission not attributable to any other cause. For subjects presenting with relapsed AML, more than 5% blasts on baseline bone marrow assessment is required. As used herein, “refractory acute myeloid leukemia” is defined as a failure to achieve a complete remission or complete remission with incomplete blood recovery after previous therapy. Any number of prior anti-leukemia schedules is allowed. As used herein, “complete remission” is defined as morphologically leukemia free state (i.e. bone marrow with less than 5% blasts by morphologic criteria and no Auer rods, no evidence of extramedullary leukemia) and absolute neutrophil count greater than or equal to 1,000/μL and platelets greater than 100,000/μL. As used herein, “complete remission with incomplete blood recovery” is defined as morphologically leukemia free state (i.e. bone marrow with less than 5% blasts by morphologic criteria and no Auer rods, no evidence of extramedullary leukemia) and neutrophil count less than 1,000/μL or platelets less than 100,000 μL in the blood.

In some embodiments, the methods described herein further comprise selecting a subject who relapses from complete remission of acute myeloid leukemia after receiving an induction chemotherapy treatment regimen.

Pharmaceutical Compositions

The disclosure contemplates compositions comprising the glutamine metabolism inhibitors described herein and at least one chemotherapeutic agent (e.g., a chemotherapeutic agent to which acute myeloid leukemia cells in a patient are or become resistant).

In some aspects, the disclosure provides a pharmaceutical composition comprising an effective amount of a glutamine metabolism inhibitor, and an effective amount of at least one chemotherapeutic agent as described herein.

In some embodiments, a pharmaceutical composition comprises an effective amount of a glutamine metabolism inhibitor, an effective amount of at least one chemotherapeutic agent, and a pharmaceutically acceptable carrier, diluent, or excipient.

The compositions comprising the glutamine metabolism inhibitor and the at least one chemotherapeutic agent can be used for treating acute myeloid leukemia as described herein. In some embodiments, the composition is useful for inducing complete remission of leukemia in the subject. In some embodiments, the composition is useful for inducing complete remission of acute myeloid leukemia in the subject. In some embodiments, the composition is useful for inducing complete remission of acute leukemia in the subject in the absence of a relapse risk due to residual leukemic cells in the subject's bone marrow or peripheral blood.

Formulation and Administration

The glutamine metabolism inhibitor and/or chemotherapeutic agent described herein can be administered alone or with suitable pharmaceutical carriers, and can be in solid or liquid form such as, tablets, capsules, powders, solutions, suspensions, or emulsions. As used herein, the term “administered” refers to the placement of an inhibitor or agent described herein, into a subject by a method or route which results in at least partial localization of the inhibitor or agent at a desired site. A glutamine metabolism inhibitor and/or chemotherapeutic agent described herein can be administered by any appropriate route which results in effective treatment in the subject, i.e. administration results in delivery to a desired location in the subject where at least a portion of the composition is delivered. For a comprehensive review on drug delivery strategies see Ho et al., Curr. Opin. Mol. Ther. (1999), 1:336-3443; Groothuis et al., J. Neuro Virol. (1997), 3:387-400; and Jan, Drug Delivery Systems: Technologies and Commercial Opportunities, Decision Resources, 1998, content of all which is incorporated herein by reference. Exemplary routes of administration of the glutamine metabolism inhibitor (e.g., DON) and/or chemotherapeutic agents described herein include, without limitation, intravenous administration, e.g., as a bolus or by continuous infusion over a period of time, intramuscular, intraperitoneal, intracerobrospinal, subcutaneous, intra-articular, intrasynovial, intrathecal, oral, topical, or inhalation routes. The glutamine metabolism inhibitor and/or chemotherapeutic agents can be formulated in pharmaceutically acceptable compositions which comprise a therapeutically-effective amount of the inhibitor and/or agent, formulated together with one or more pharmaceutically acceptable carriers (additives) and/or diluents, or excipients. The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171.

In some embodiments, the glutamine metabolism inhibitor and/or chemotherapeutic agents described herein can be administrated encapsulated within a nanoparticle (e.g., a lipid nanoparticle). In some embodiments, glutamine metabolism inhibitors and/or chemotherapeutic agents described herein can be administered encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., endothelial cells) can also be used as pharmaceutically acceptable carriers.

The glutamine metabolism inhibitor and/or chemotherapeutic agents can be administrated to a subject in combination with other pharmaceutically active agents. Exemplary pharmaceutically active agents include, but are not limited to, those found in Harrison's Principles of Internal Medicine, 13^{th} Edition, Eds. T. R. Harrison et al. McGraw-Hill N.Y., NY; Physician's Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference. In some embodiments, the pharmaceutically active agent is a conventional treatment for acute myeloid leukemia. In some embodiments, the pharmaceutically active agent is a conventional treatment for an autoimmune or inflammatory condition. The skilled artisan will be able to select the appropriate conventional pharmaceutically active agent for treating any particular disease or disease subtype using the references mentioned above based on their expertise, knowledge and experience.

The glutamine metabolism inhibitor, chemotherapeutic agent, and/or the other pharmaceutically active agent can be administrated to the subject in the same pharmaceutical composition or in different pharmaceutical compositions (at the same time or at different times). For example, a glutamine metabolism inhibitor and at least one chemotherapeutic agent can be formulated in the same composition or in different compositions.

The pharmaceutical compositions can be included in a container, pack, or dispenser together with instructions for administration.

As used herein, “effective amount”, “effective amounts”, or “therapeutically effective amounts” means an amount of the agent (e.g., glutamine metabolism inhibitor) which is effective to eradicate a majority or all of the leukemic cells (e.g., stem or progenitor cells) in a population of cells or a subject. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in the acute myeloid leukemia or autoimmune or inflammatory disorder.

Kits

The glutamine metabolism inhibitor and/or chemotherapeutic agents described herein can be provided in a kit. The kit includes (a) the glutamine metabolism inhibitor, e.g., a composition that includes the glutamine metabolism inhibitor, (b) the at least one chemotherapeutic agent, and (c) informational material. The informational material can be descriptive, instructional, marketing or other material that relates to the methods described herein and/or the use of the inhibitors and agents for the methods described herein. For example, the informational material describes methods for administering the glutamine metabolism inhibitors and chemotherapeutic agents to a subject for treating acute myeloid leukemia.

The informational material can include instructions to administer the glutamine metabolism inhibitors and chemotherapeutic agents described herein in a suitable manner, e.g., in a suitable dose, dosage form, or mode of administration. In some embodiments, the instructions recommend administering an effective amount of a glutamine metabolism inhibitor (e.g., DON). In some embodiments, the instructions recommend administering a glutamine metabolism inhibitor in an amount of 0.3 mg/kg once daily for 5 days. The informational material can include instructions for selecting a suitable subject, e.g., a human, e.g., a human suffering from relapsed or refractory acute myeloid leukemia. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the inhibitor and/or its use in the methods described herein. Of course, the informational material can also be provided in any combination of formats.

In addition to the glutamine metabolism inhibitor and the at least one chemotherapeutic agent, the kit can include other ingredients, such as a solvent or buffer, a stabilizer or a preservative, and/or an agent for treating a condition or disorder described herein, e.g. acute myeloid leukemia. Alternatively, the other ingredients can be included in the kit, but in different compositions or containers than the glutamine metabolism inhibitor and the chemotherapeutic agent. In such embodiments, the kit can include instructions for admixing the glutamine metabolism inhibitor, the chemotherapeutic agent, and the other ingredients, or for using the glutamine metabolism inhibitor and the chemotherapeutic agent together with the other ingredients.

The glutamine metabolism inhibitor described herein can be provided in any form, e.g., liquid, dried or lyophilized form. It is preferred that the glutamine metabolism inhibitor be substantially pure and/or sterile. When the glutamine metabolism inhibitor is provided in a liquid solution, the liquid solution preferably is an aqueous solution, with a sterile aqueous solution being preferred. When the glutamine metabolism inhibitor is provided as a dried form, reconstitution generally is by the addition of a suitable solvent. The solvent, e.g., sterile water or buffer, can optionally be provided in the kit.

The kit can include one or more containers for the composition containing the glutamine metabolism inhibitor and the chemotherapeutic agent(s). In some embodiments, the kit contains separate containers, dividers or compartments for the glutamine metabolism inhibitor (e.g., in a composition), the chemotherapeutic agent, and informational material. For example, the glutamine metabolism inhibitor and the chemotherapeutic agent can each be contained in a bottle, vial, or syringe, and the informational material can be contained in a plastic sleeve or packet. In other embodiments, the separate elements of the kit are contained within a single, undivided container. For example, the glutamine metabolism inhibitor (e.g., in a composition) and the chemotherapeutic agent are contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the glutamine metabolism inhibitor (e.g., in a composition) and the chemotherapeutic agent. For example, the kit includes a plurality of syringes, ampules, foil packets, or blister packs, each containing a single unit dose of the agent. The containers of the kits can be air tight and/or waterproof.

In some aspects, a kit comprises: a glutamine metabolism inhibitor, at least one chemotherapeutic agent, and instructions for administering the glutamine metabolism inhibitor and the at least one chemotherapeutic agent to a subject suffering from acute myeloid leukemia.

In some embodiments, the instructions further comprise directions for administering the at least one chemotherapeutic agent as part of an induction chemotherapy treatment regimen for the subject.

In some embodiments, the instructions further comprise directions for administering the glutamine metabolism inhibitor, and the at least one therapeutic agent to induce complete remission of acute myeloid leukemia in the subject.

In some embodiments, the instructions further comprise directions for administering the glutamine metabolism inhibitor, and the at least one therapeutic agent to induce complete remission of acute myeloid leukemia in the subject, without risk of relapse by completely eradicating leukemic cells in the subject.

Agents

Without wishing to be bound by any theory, the agents (e.g., glucose metabolism inhibitors) disclosed herein inhibit glutamine metabolism. Accordingly, while certain aspects of the invention relate to the use of certain glutamine metabolism inhibitors (e.g., DON and analogs thereof), it should be understood that the present inventions are not limited to such glutamine metabolism inhibitors. Rather, contemplated herein are any means of interfering with glutamine metabolism and thereby eradicating leukemic cells (e.g., chemoresistant leukemic cells).

For example, in certain aspects, the methods, kits and compositions disclosed herein may comprise any agents or compositions that are capable of or useful for inhibiting glutamine metabolism. Exemplary types of agents that can be used as glutamine metabolism inhibitors include small organic or inorganic molecules; saccharines; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof. In some aspects, the glutamine metabolism inhibitor is 6-diano-5-oxo-L-norleucine (DON) or analogs thereof.

The disclosure contemplates the use of an agent in combination with at least one additional chemotherapeutic agent, such as a chemotherapeutic agent, in the methods, compositions, and kits described herein. The disclosure contemplates the use of any chemotherapeutic agent that is useful for treating cancer (e.g., leukemia). Exemplary chemotherapeutic agents that can be administered in combination with the glutamine metabolism inhibitor of the present invention include alkylating agents (e.g. cisplatin, carboplatin, oxaloplatin, mechlorethamine, cyclophosphamide, chorambucil, nitrosureas); anti-metabolites (e.g. methotrexate, pemetrexed, 6-mercaptopurine, dacarbazine, fludarabine, 5-fluorouracil, arabinosycytosine, capecitabine, gemcitabine, decitabine); plant alkaloids and terpenoids including vinca alkaloids (e.g. vincristine, vinblastine, vinorelbine), podophyllotoxin (e.g. etoposide, teniposide), taxanes (e.g. paclitaxel, docetaxel); topoisomerase inhibitors (e.g. notecan, topotecan, amasacrine, etoposide phosphate); antitumor antibiotics (dactinomycin, doxorubicin, epirubicin, and bleomycin); ribonucleotides reductase inhibitors; antimicrotubules agents; and retinoids. (See, e.g., Cancer: Principles and Practice of Oncology (V. T. DeVita, et al., eds., J.B. Lippincott Company, 9^{th} ed., 2011; Brunton, L., et al. (eds.) Goodman and Gilman's The Pharmacological Basis of Therapeutics, 12^{th} Ed., McGraw Hill, 2010).

The compositions, methods, and kits described herein contemplate the use of at least one chemotherapeutic agent, particularly one to which AML cells in a patient are or become resistant (e.g., by any resistance mechanism). In some embodiments, the at least one chemotherapeutic agent comprises an antimetabolite agent. In some embodiments, the at least one chemotherapeutic agent comprises cytarabine. In some embodiments, the at least one chemotherapeutic agent comprises an anthracycline agent. In some embodiments, the at least one chemotherapeutic agent comprises doxorubicin. In some embodiments, the at least one chemotherapeutic agent comprises an antimetabolite agent and an anthracycline agent. In some embodiments, the at least one chemotherapeutic agent comprises cytarabine and the anthracycline agent comprises doxorubicin. It should be appreciated that administration of a glutamine metabolism inhibitor described herein (e.g., DON) selectively targets leukemic cells by, in part, overcoming chemoresistance exhibited by leukemic cells, such as glutamine metabolism-mediated chemoresistance.

Biomarkers

The disclosure contemplates the use of one or more glutamine transporters as biomarkers for identifying chemoresistant leukemia (e.g., AML) cells. The expression of various glutamine transporters increases in AML cells after the cells are treated with chemotherapy, and therefore they may act as biomarkers for identifying chemoresistant cells.

In some aspects, the disclosure provides methods for detecting chemoresistant leukemia (e.g., AML) cells. In some embodiments, a sample (e.g., a biological sample) is obtained from a subject and the sample is assessed to determine if one or more glutamine transporters are present. The sample may be obtained from a subject who has previously been treated with chemotherapy, or who is currently being treated with chemotherapy.

A sample obtained from a subject may be assayed to detect the presence of one or more biomarkers which would signify the presence of chemoresistant AML cells. For example, a flow panel assay is applied to a sample to detect the level or activity of one or more glutamine transporter gene signatures that signify the presence of chemoresistant AML cells. In some embodiments, the sample is assessed to measure mRNA levels of one or more glutamine transporters. In some embodiments, the one or more glutamine transporters are selected from the group consisting of Slc5a1, Slc38a1, and Slc38a2. In some embodiments, the methods for detecting chemoresistant leukemia cells further includes detecting increased protein levels of SLC38A1 in AML cells.

In some embodiments, methods described herein may be used to detect residual chemoresistant AML cells with high accuracy. The improved detection of residual chemoresistant cells can further improve quantification of minimal residual disease (MRD), and therefore allow clinical personnel to make better decisions about patient follow-up post treatment.

### Some Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, kits and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, kits and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following example illustrates some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

Acute myeloid leukemia (AML) is one of the most challenging cancers to treat. Induction chemotherapy (iCT) remains the standard of care, but the incidence of refractory and relapsed AML is high. Unraveling the molecular regulation of this chemoresistance is critical to provide new treatment options for patients. Cellular metabolism plays a central role in the control of cell fate, and a dysregulated metabolism is now widely accepted as a hallmark of many cancers. It was hypothesized that chemoresistance in AML arises at the time of maximal iCT response, with the residual cells manifesting distinctive metabolic features that enable their survival under the extreme stress of chemotherapy.

Visualizing the Moment of Maximal Response to Chemotherapy in AML

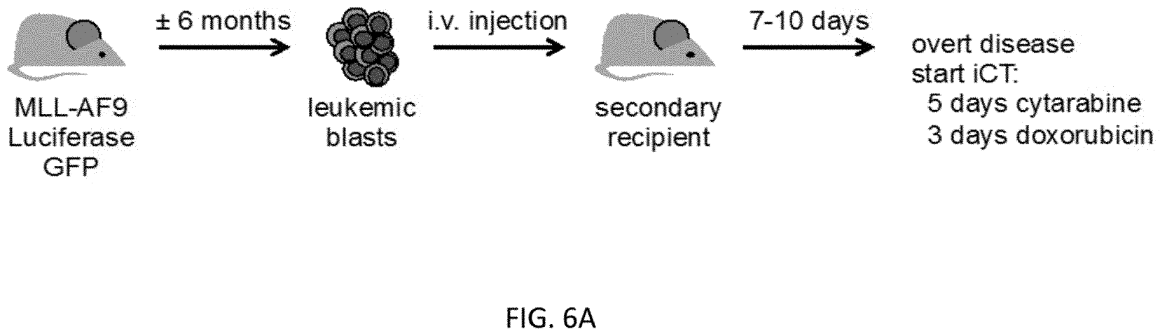

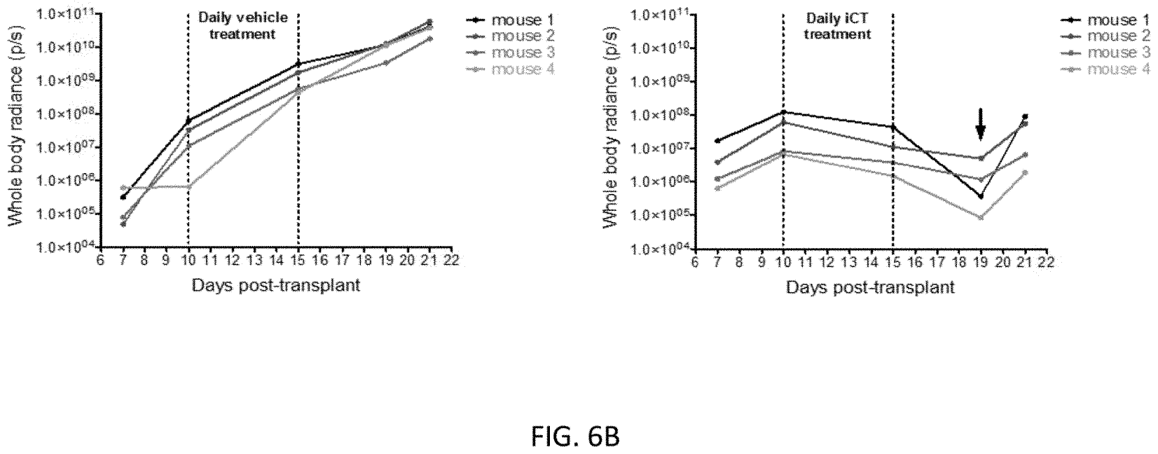

To test this hypothesis, a mouse model was used in which cells express MLLAF9, driving leukemia development, luciferase, and GFP. The mouse model allows real-time monitoring of leukemic burden through bioluminescence imaging, and therefore identification of the moment of maximal response to iCT. Bone marrow cells derived from terminally ill primary mice were intravenously transplanted into secondary wildtype recipients, leading to the development of a very aggressive disease that can be monitored in real time since only AML cells express luciferase and GFP. Mice were treated with a chemotherapy regimen that closely mimics the one used in patients (cytarabine for 5 days and doxorubicin for the first 3 days) or vehicle, and followed disease progression using bioluminescence imaging (FIG. 6A). It was discovered that the amount of maximal response occurs 3-4 days after the last dose of chemotherapy (FIG. 6B). This means that at this moment, after the selection pressure of chemotherapy, massive neighboring cell death, and possibly other stress factors such as niche alteration, certain cells have adapted and are now started to grow again.

Optimization of Untargeted Metabolomics Analysis of Freshly Isolated AML Cells

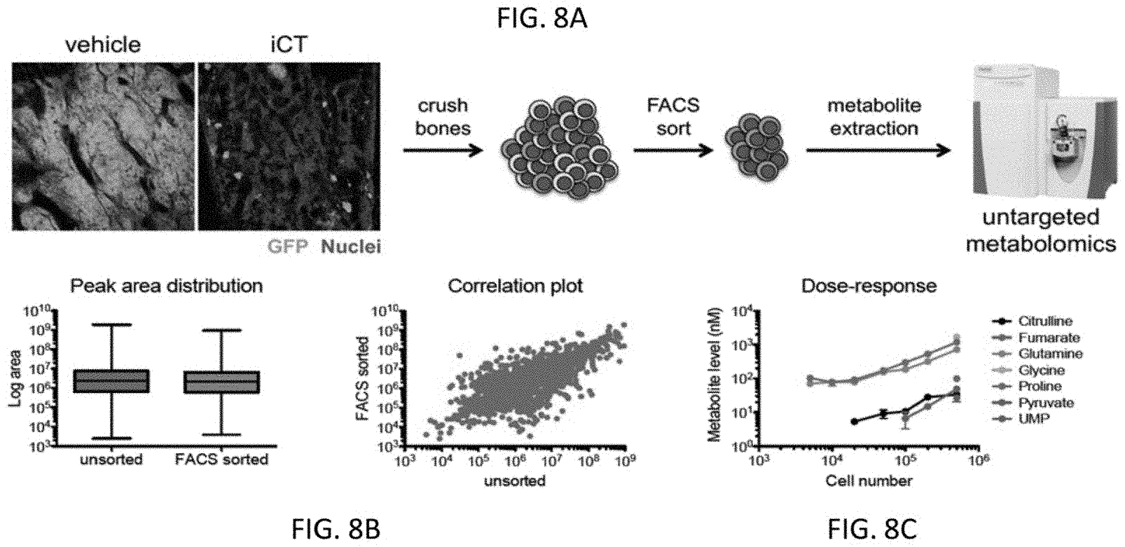

GFP-expressing AML cells were isolated from bone marrow of mice receiving treatment with vehicle or iCT (cytarabine for 5 days and doxorubicin for the first 3 days), both at the moment of maximal response (3 days after last dose of iCT) and after relapse (10 days after last dose). Since cellular metabolism is highly dynamic and sensitive to environmental perturbations, an optimized methodology was developed to analyze the metabolome of freshly isolated AML cells. As seen in FIG. 8A, long bones of the mice were dissected, crushed, and GFP⁺ AML cells were isolated using FACS. All steps were performed at 4° C. to minimize metabolic changes. Next, cells were lysed and polar metabolites were obtained after methanol:chloroform extraction. Data was acquired on a ThermoFisher Q-Exactive LC-MS with Zic-pHILIC column in an untargeted manner, and peak identification was performed using CompoundDiscoverer 2.0.

The effect of FACS sorting on the cellular metabolome was assessed using AML cells in culture (FIG. 8B), which were either used for immediate metabolite extraction (unsorted), or subjected to incubation at 4° C. and FACS sorting prior to metabolite extraction (FACS sorted). No significant differences were seen in the peak area distribution, and good correlation (R²=0.59) between the levels of individual metabolites was observed. To determine the number of cells needed to obtain sufficient coverage of the cellular metabolome a dose-response experiment was performed (FIG. 8C), which showed that while some metabolites were detectable at lower cell numbers, 25,000 cells are needed for the detection of several metabolites involved in central carbon metabolism.

Metabolic Profiling of AML Cells Reveals Changes in Glutamine Metabolism after Chemotherapy

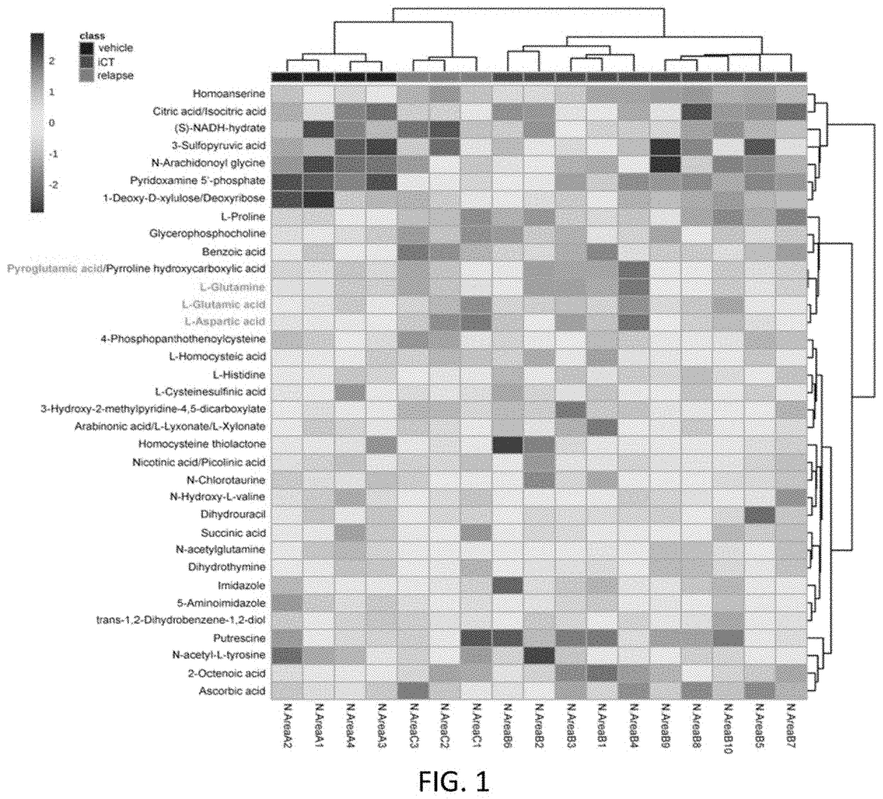

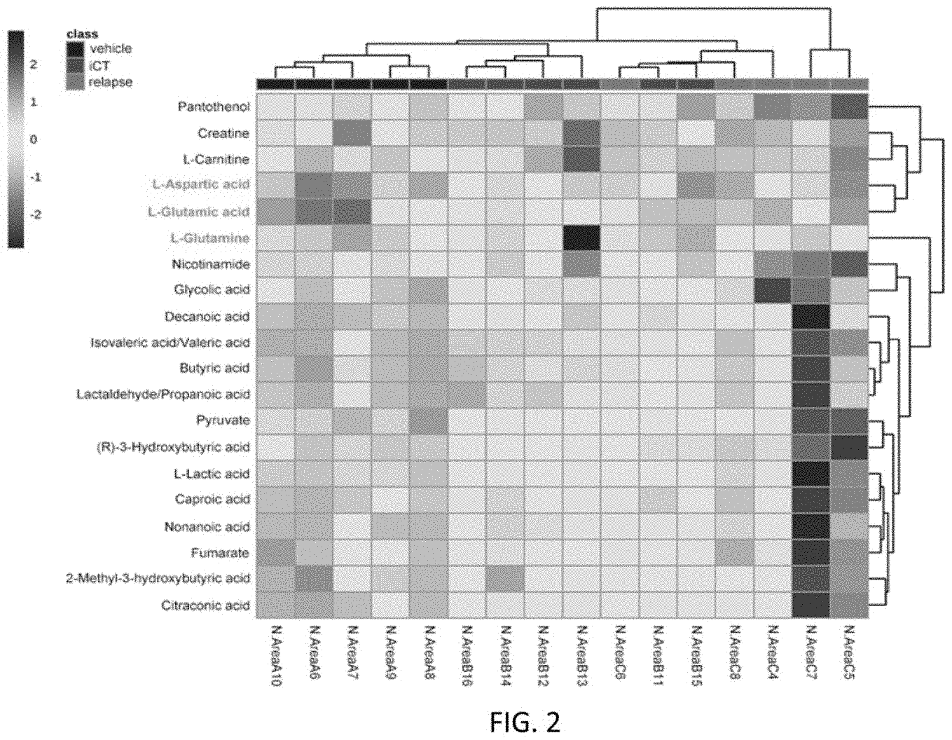

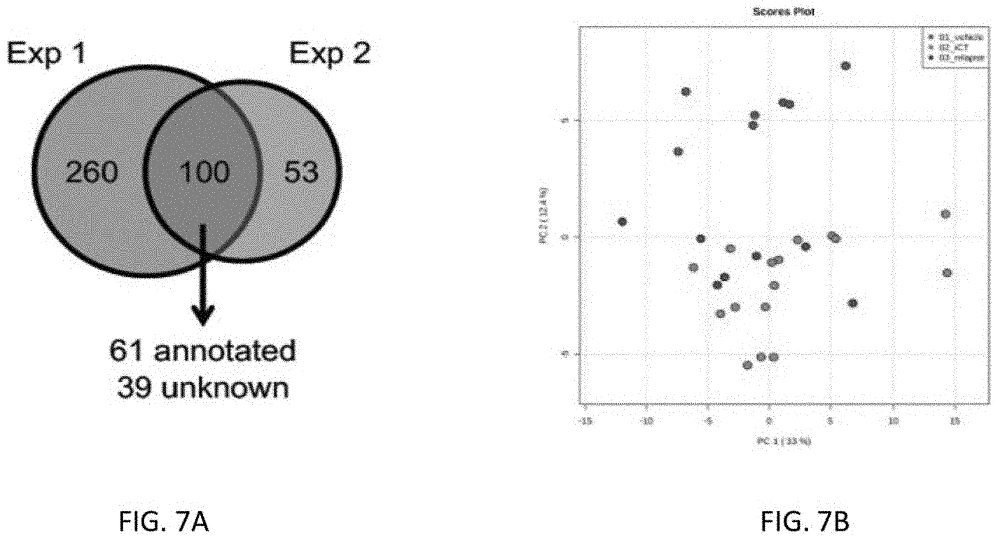

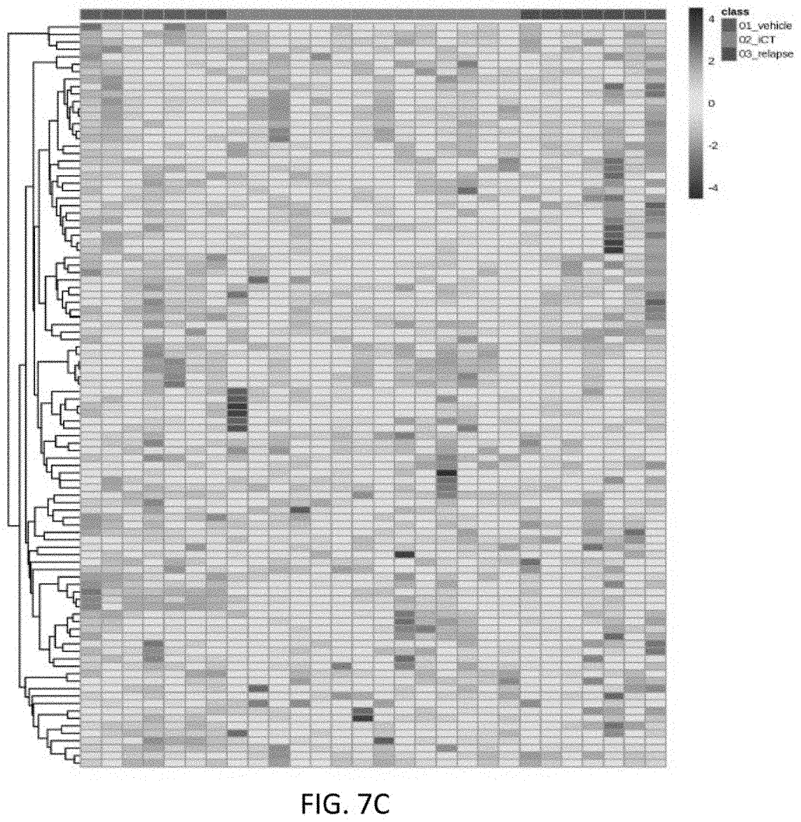

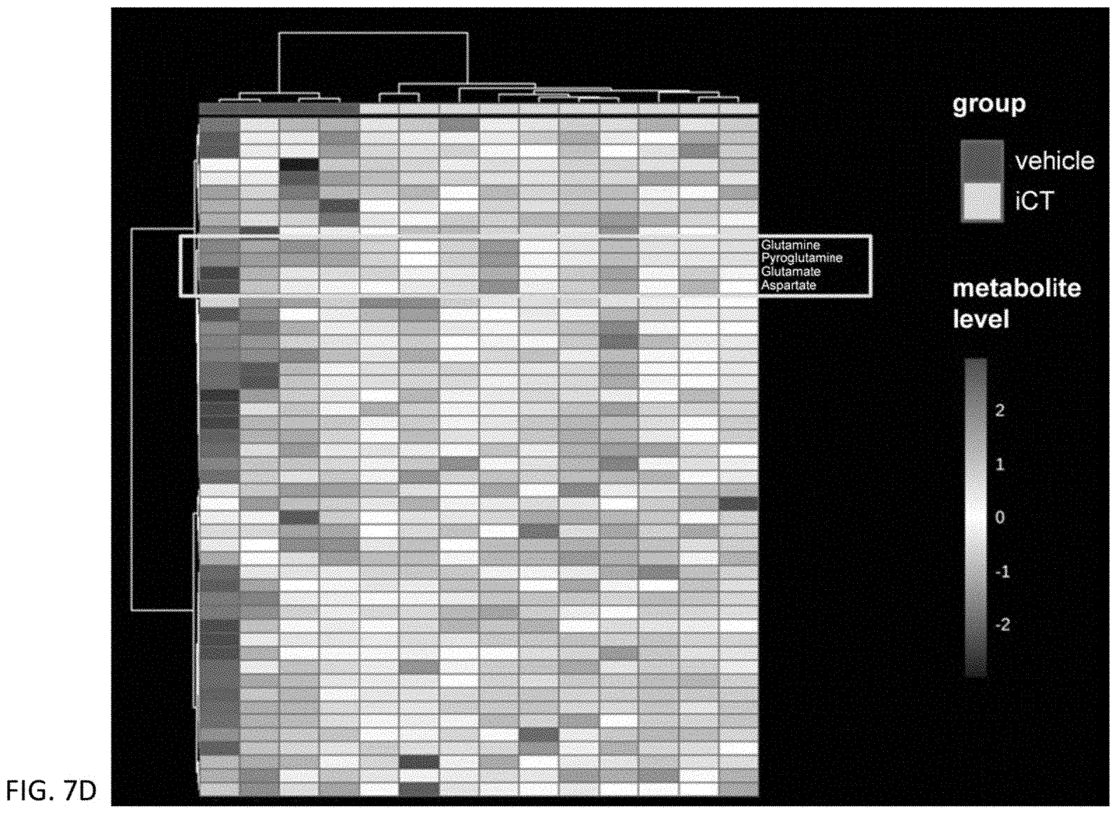

A platform for untargeted metabolomics analysis of freshly sorted cells was developed, which allows us to measure the levels of more than 670 metabolites using 500,000 cells per sample. In a first experiment, 4 vehicle treated mice and 15 iCT-treated mice were used. For the iCT-treated mice, some samples had to be pooled in order to obtain sufficient cells for analysis. Analysis of 25,000 cells allowed detection of 360 metabolites, of which 216 (60%) could be putatively identified (FIG. 7D). Of the 360 metabolites that were measured, only very few differed between the two groups. And of the ones that were different, many are unknown metabolites. However, one known pathway stood out: glutamine metabolism. Multiple metabolites involved in glutamine metabolism were identified, and 4 metabolites: glutamine, glutamate, aspartate and pyroglutamate, were all significantly increased in the chemoresistant cells (FIGS. 1-2, 7D).

More specifically, the methodology for ex vivo untargeted metabolomics was used to identify the metabolic profile of AML cells freshly isolated from the bone marrow of mice treated with vehicle (vehicle group) or treated with iCT, both at 3 days after the last dose (iCT group) or at 10 days after the last dose (relapse group). Putative metabolite annotation was performed using the MzCloud database, Human Metabolome Database and the KEGG pathway database, and data visualization and analysis was performed using the MetaboAnalyst 3.0 software package. Two independent experiments were performed, and metabolites detected in both experiments (61 putatively annotated, 39 unknown) were used for subsequent analysis (FIG. 7A). Principal component analysis showed separation of the vehicle group from the iCT and relapse groups (FIG. 7B), and heatmap-based visualization of the results revealed sets of metabolites exhibiting different patterns between the three groups (FIG. 7C). Statistical analysis (1-way ANOVA with Tukey's HSD post-hoc test) revealed significant differences between the groups in 24 of the 100 metabolites.

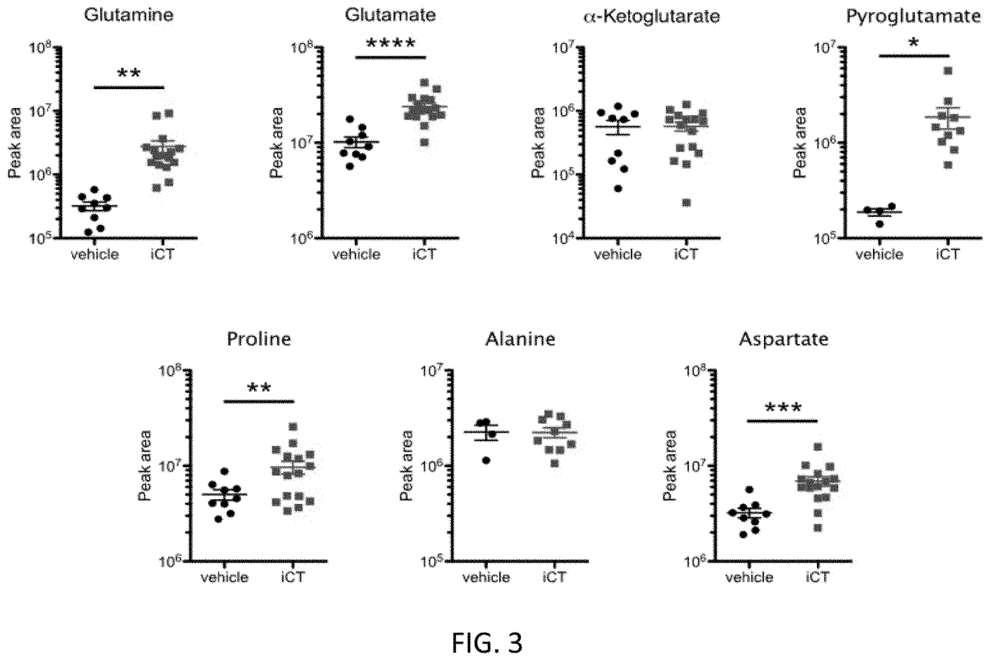

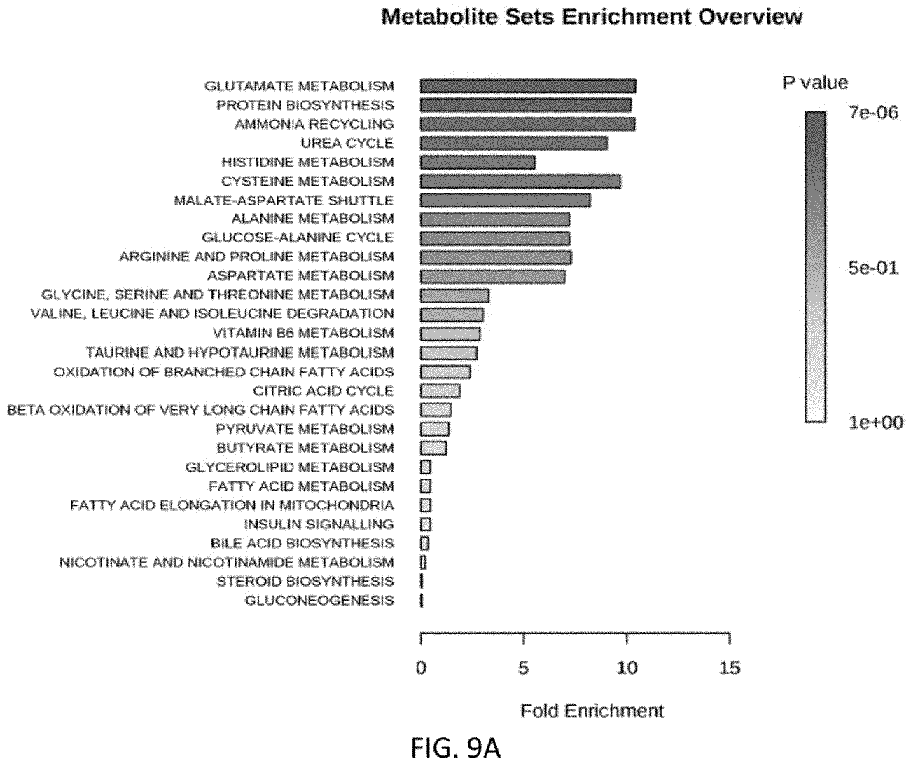

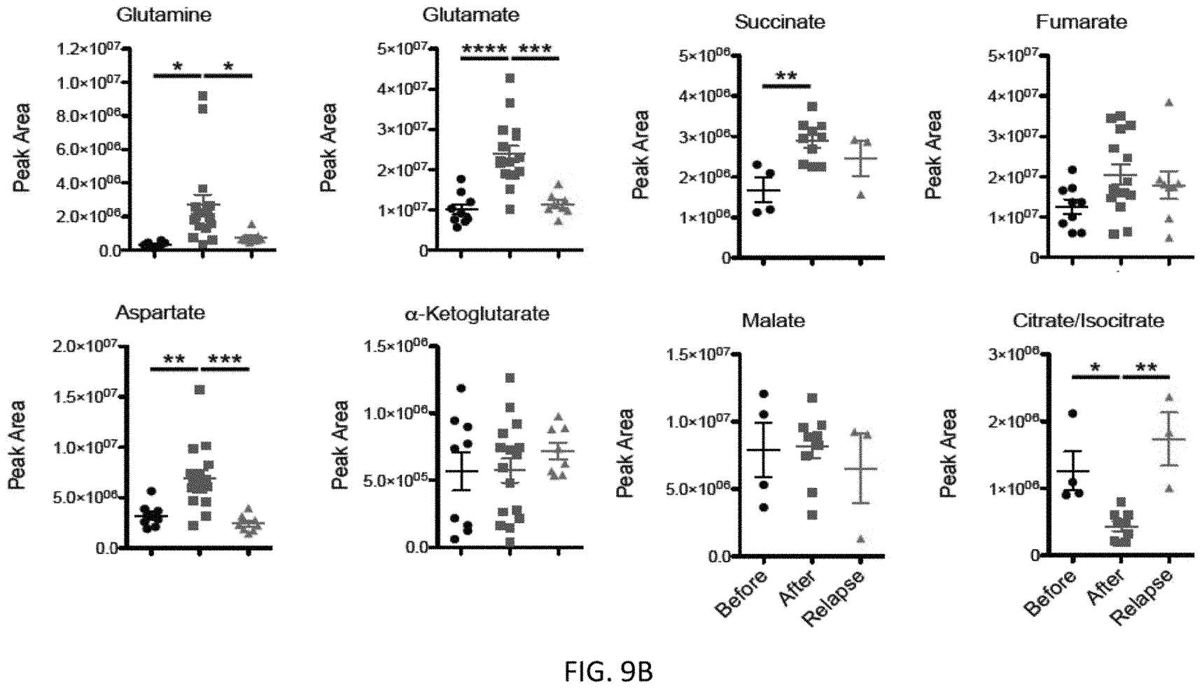

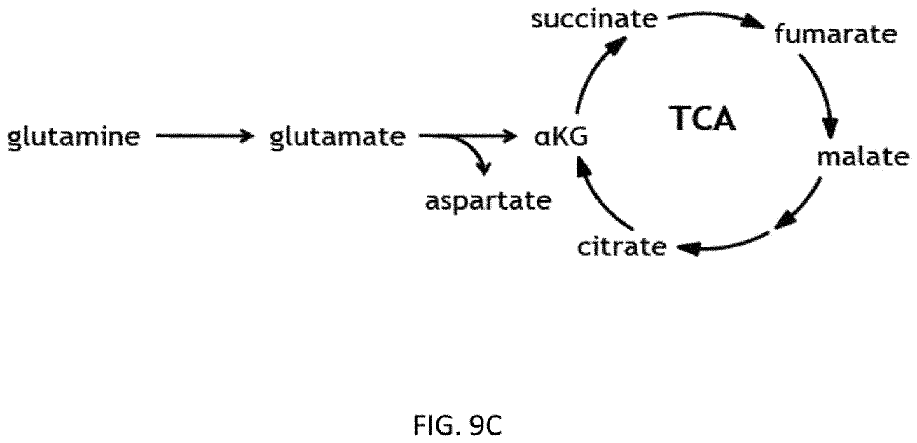

Data for the 61 annotated metabolites of the vehicle and iCT groups (FIG. 7A) were further analyzed using the enrichment analysis module of the MetaboAnalyst software (FIG. 9A). The top four pathways all contained the same three metabolites that drove the enrichment and statistical significance, glutamine, glutamate, and asparate, which form the core of glutamine metabolism (FIG. 9B). When analyzing the levels of these metabolites in the three groups, a similar pattern was revealed: low in the vehicle group, high in the iCT group and low again in the relapse group (FIG. 9C), suggesting a dynamic role for glutamine metabolism in the immediate stress response to iCT (FIG. 3 and FIG. 9B). TCA cycle metabolites (downstream of glutamine metabolism) showed overall less differences between the groups, although succinate levels were increased while citrate/isocitrate levels were decreased in chemoresistant AML cells.

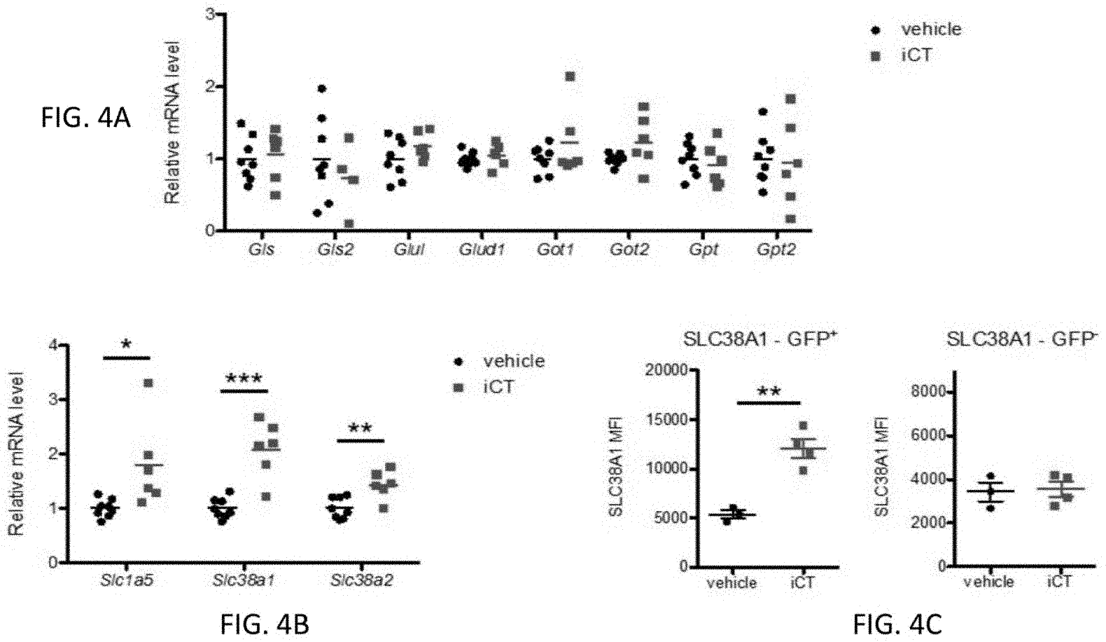

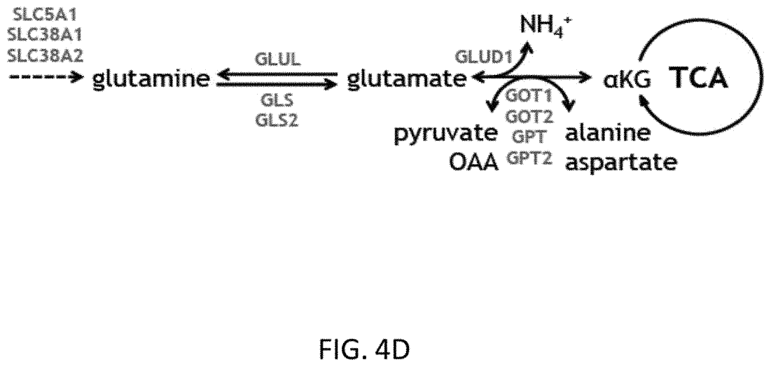

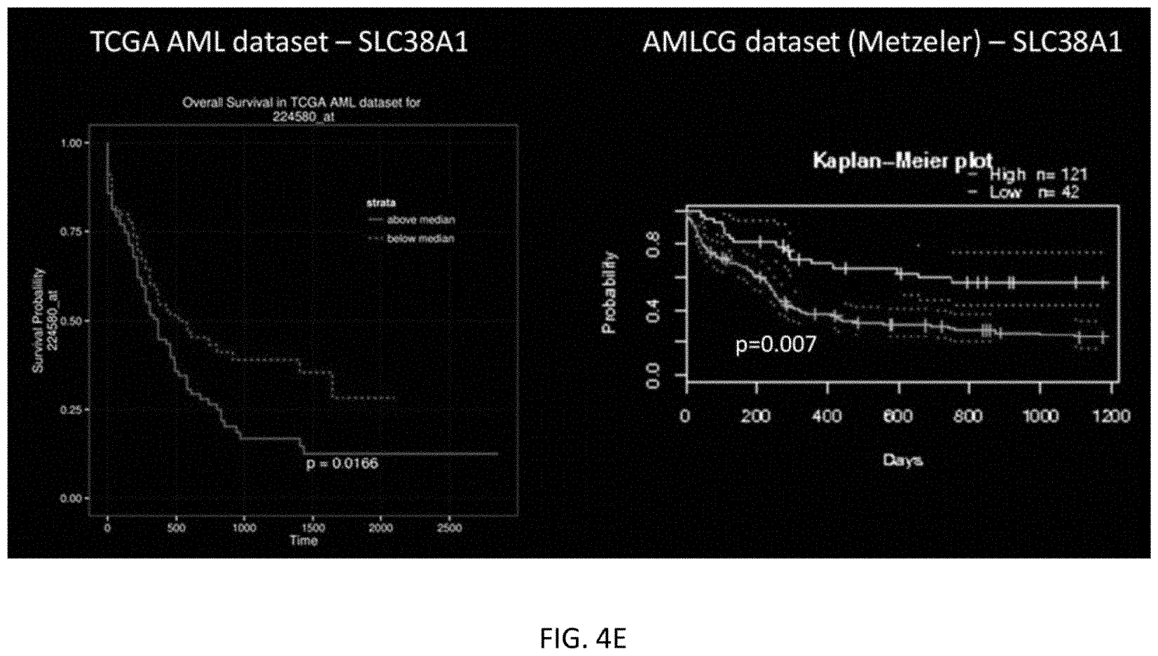

Glutamine plays several key roles in cellular metabolism (FIGS. 4D and 9B). After being taken up in the cells, glutamine can be used as an amino acid for protein synthesis, or it can be metabolized by conversion into glutamate through the action of different enzymes. The amide group that is released in this conversion can be released as ammonia, or it can be used for nucleotide synthesis or for glycosylation. Glutamate can then be further metabolized in different ways. Its carbon backbone can be used for the production of the antioxidant glutathione, or for proline synthesis. Glutamate can also be converted into alpha-ketoglutarate, an intermediate of the mitochondrial TCA cycle. The amine group that is released in this conversion can again be released as ammonia, or it can be used for the synthesis of other amino acids such as alanine and aspartate. It was seen that many metabolites in this pathway were altered (FIG. 9C). Not only were glutamine and glutamate increased in chemoresistant cells, but proline, aspartate, and pyroglutamate (a breakdown/recycling product of glutathione) were also increased.

Changes in Glutamine Metabolism are not Reflected in Enzyme Expression Levels

To further explore the role of glutamine metabolism in the acquisition of chemoresistance in AML cells, analysis of the transcriptomic profile of vehicle- and iCT-treated AML cells (RNAseq) showed that expression of the majority of genes encoding for enzymes involved in glutamine metabolism did not differ between groups (FIGS. 4A, 4D). In contrast, mRNA levels of several glutamine transporters, including Slc1a5, Slc38a1, and Slc38a2, were increased in chemoresistant AML cells (FIG. 4B). Flow cytometric analysis further showed increased protein levels of SLC38A1 in AML (GFP+) cells in the bone marrow of mice treated with iCT, a change that was not seen in normal hematopoietic (GFP−) cells (FIG. 4C). This shows that normal and leukemic cells respond differently to chemotherapy, and suggests that the changes in glutamine metabolism in response to chemotherapy are specific to AML cells. Interestingly, when looking in AML patient survival datasets, patients that had leukemia with high expression levels of SLC38A1 had lower survival probability (FIG. 4E).

Inhibition of Glutamine Metabolism Increases Chemosensitivity in AML Cells

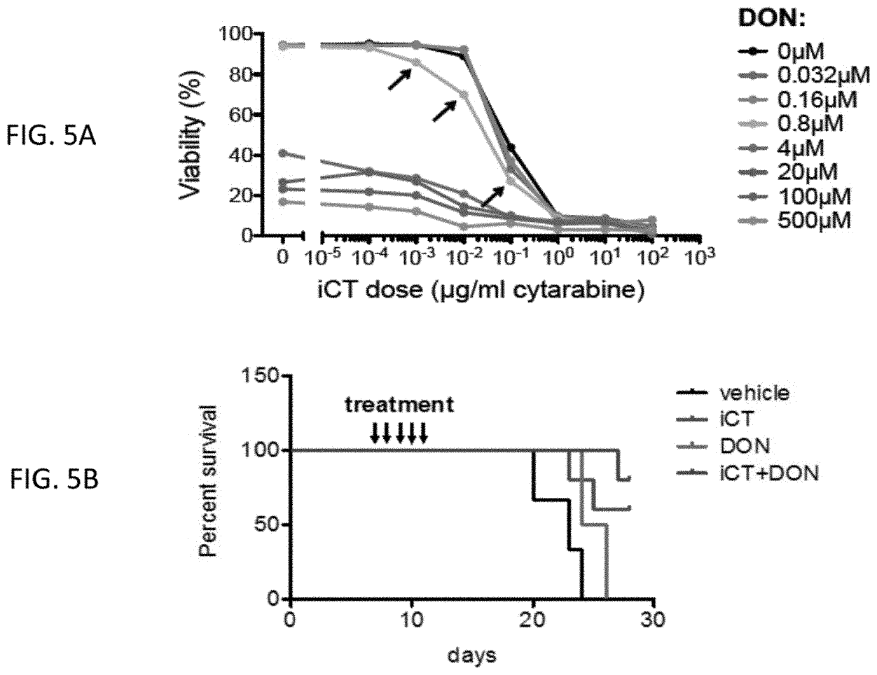

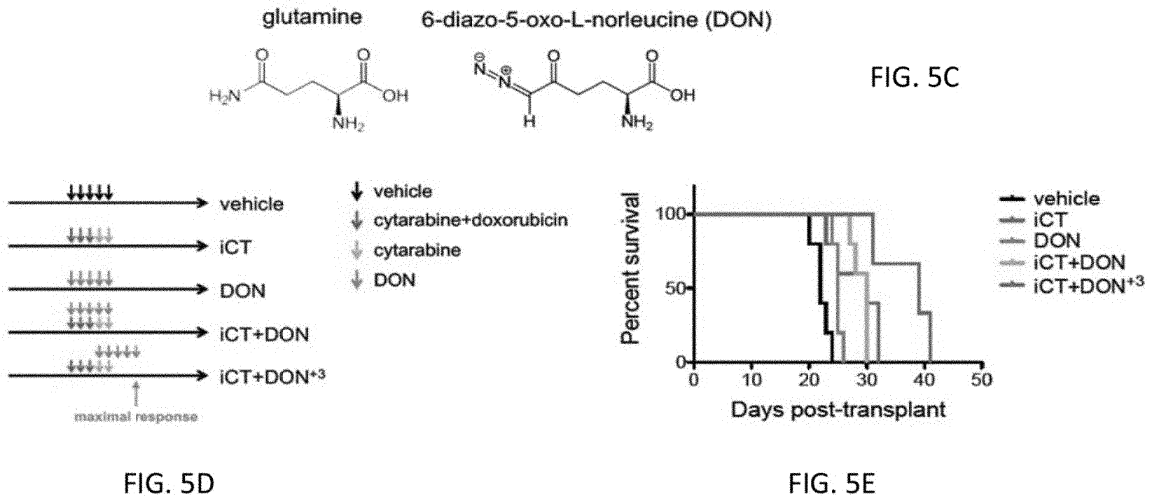

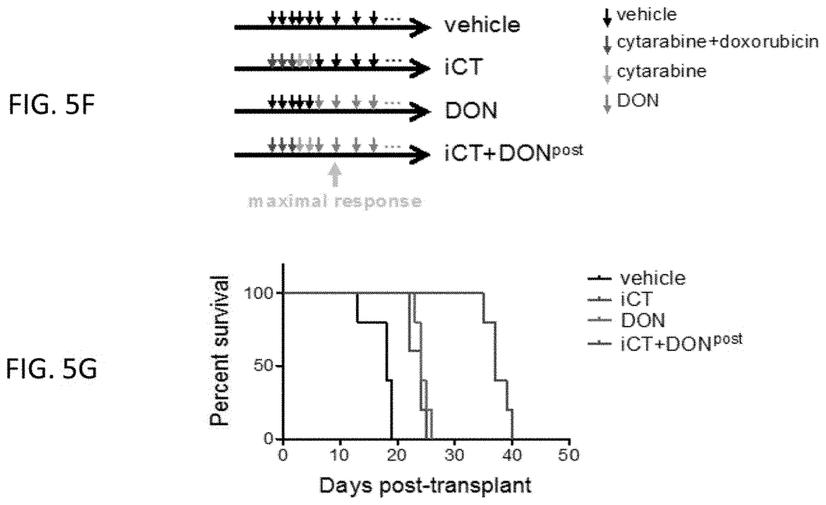

To confirm that glutamine metabolism plays a functional role in protecting AML cells from chemotherapy, human AML cells (THP1) were treated in culture with iCT in combination with 6-diazo-5-oxo-L-norleucine (DON), a glutamine analog and antagonist that inhibits all glutamine-dependent enzymes (FIG. 5C). Treatment of the THP1 human AML cell line with different doses of iCT (cytarabine+doxorubicin) in combination with DON revealed synergy at several doses (black arrows) in vitro (FIG. 5A). Mice carrying AML were then treated in combination with DON at different regimens (FIG. 5D). A short treatment with DON (daily for 5 days at 0.3 mg/kg) did not have much effect, and when DON was given together with iCT, survival of the mice was similar to the treatment with iCT alone and DON did not extend survival (FIGS. 5B and 5E). However, when DON was delayed 3 days, spanning the moment of maximal response, a clear survival benefit was achieved (FIG. 5E). While daily injections of DON proved lethal when extended for more than 5 days, treatment was extended by injecting DON every other day at 0.3 mg/kg and survival of the mice increased substantially over treatment with iCT alone (p=0.0019) (FIG. 5F). This treatment regimen induced even greater survival benefit, and showed clear synergism between iCT and DON treatment (FIG. 5G). These data show that activation of glutamine metabolism protects AML cells at the moments of maximal stress, and reveal the potential of targeting glutamine metabolism to enhance the response to chemotherapy.

### CONCLUSIONS

These results highlight the power of using untargeted metabolomics to uncover novel chemoprotective metabolic pathways, and underscore the uniqueness of the approach as glutamine metabolism would not have been picked up through transcriptomics analysis alone. In addition, several currently unknown metabolites were identified, of which levels differed significantly in chemoresistant AML cells. Taken together, the findings provide insight into the metabolic programs that determine chemoresistance in vivo and indicate that targeting glutamine metabolism provides a basis for overcoming chemoresistance in AML.

1. A method of treating acute myeloid leukemia in a subject in need thereof, the method comprising administering to the subject an effective amount of a glutamine metabolism inhibitor and an induction chemotherapy treatment (iCT) regimen, thereby treating acute myeloid leukemia in the subject,
wherein the iCT regimen comprises administering cytarabine and doxorubicin to the subject for a period of 3 days, followed by administering cytarabine alone to the subject for a period of 2 days, and
wherein the glutamine metabolism inhibitor is administered for a period of at least 5 days beginning the day after completing the iCT regimen.

2. The method of claim 1, wherein the glutamine metabolism inhibitor is administered every other day.

3. The method of claim 2, wherein the glutamine metabolism inhibitor is administered for a period of at least 10 days.

4. The method of claim 1, wherein the glutamine metabolism inhibitor comprises a small molecule inhibitor.

5. The method of claim 1, wherein the glutamine metabolism inhibitor comprises a glutaminase inhibitor.

6. The method of claim 1, wherein the glutamine metabolism inhibitor comprises a GSL1 inhibitor and/or a GSL2 inhibitor, or
wherein the glutamine metabolism inhibitor comprises 6-diano-5-oxo-L-norleucine (DON) or an analog thereof.

7. (canceled)

8. The method of claim 1, wherein the glutamine metabolism inhibitor comprises a solute carrier family 38 member 1 (SLC38a1) inhibitor, a solute carrier family 38 member 2 (SLC38a2) inhibitor, glutamate-cysteine ligase (GCL) inhibitor, a solute carrier family 7 member 11 (SLC7A11) inhibitor, or a dihydroorotate dehydrogenase (DHODH) inhibitor.

9.-12. (canceled)

13. The method of claim 1, wherein the administration of the glutamine metabolism inhibitor and the induction chemotherapy treatment regimen results in reduced expression of one or more glutamine transporters, as compared to administering only induction chemotherapy.

14. The method of claim 13, wherein the one or more glutamine transporters are selected from the group consisting of Slc5a1, Slc38a1, and Slc38a2.

15. The method of claim 1, wherein the subject is suffering from refractory or relapsed acute myeloid leukemia,
wherein the subject is a subject who relapses from complete remission of acute myeloid leukemia after induction chemotherapy,
wherein treating acute myeloid leukemia comprises inducing complete remission of acute myeloid leukemia in the subject,
wherein treating acute myeloid leukemia comprises inducing complete remission of acute myeloid leukemia in the subject in the absence of a relapse risk due to residual leukemic cells in the subject's bone marrow or peripheral or
further comprising evaluating the subject to determine if the subject has refractory or relapsed acute myeloid leukemia.

16.-19. (canceled)

20. A method of treating acute myeloid leukemia in a subject in need thereof, the method comprising administering to the subject an effective amount of a glutamine metabolism inhibitor and an induction chemotherapy treatment regimen, thereby treating acute myeloid leukemia in the subject,
wherein the iCT regimen comprises administering cytarabine and doxorubicin to the subject for a period of 3 days, followed by administering cytarabine alone to the subject for a period of 2 days, and
wherein the glutamine metabolism inhibitor is administered for a period of at least 5 days with administration beginning 4 days after starting the iCT regimen.

21. The method of claim 20, wherein the glutamine metabolism inhibitor is administered every other day.

22. The method of claim 21, wherein the glutamine metabolism inhibitor is administered for a period of at least 10 days.

23. The method of claim 20, wherein the glutamine metabolism inhibitor comprises a small molecule inhibitor.

24. The method of claim 20, wherein the glutamine metabolism inhibitor comprises a glutaminase inhibitor.

25. The method of claim 20, wherein the glutamine metabolism inhibitor comprises a GSL1 inhibitor and/or a GSL2 inhibitor, or
wherein the glutamine metabolism inhibitor comprises 6-diano-5-oxo-L-norleucine (DON) or an analog thereof.

26. (canceled)

27. The method of claim 20, wherein the glutamine metabolism inhibitor comprises a solute carrier family 38 member 1 (SLC38a1) inhibitor, a solute carrier family 38 member 2 (SLC38a2) inhibitor, glutamate-cysteine ligase (GCL) inhibitor, a solute carrier family 7 member 11 (SLC7A11) inhibitor, or a dihydroorotate dehydrogenase (DHODH) inhibitor.

28.-31. (canceled)

32. The method of claim 20, wherein the administration of the glutamine metabolism inhibitor and the induction chemotherapy treatment regimen results in reduced expression of one or more glutamine transporters, as compared to administering only induction chemotherapy.

33. The method of claim 32, wherein the one or more glutamine transporters are selected from the group consisting of Slc5a1, Slc38a1, and Slc38a2.

34.-43. (canceled)

44. A method of detecting chemoresistant AML cells in a subject, comprising obtaining a sample from the subject and detecting one or more gene signatures in a sample, wherein the one or more gene signatures is selected from the group consisting of Slc5a1, Slc38a1, or Slc38a2, and wherein the presence of the gene signature indicates the presence of chemoresistant AML cells.

45.-48. (canceled)

49. A pharmaceutical composition comprising an effective amount of a glutamine metabolism inhibitor, an effective amount of at least one chemotherapeutic agent, and a pharmaceutically acceptable carrier, diluent, or excipient.

50.-59. (canceled)
